SEQUENCE LISTING (22 sequences)

SEQ ID NO: 1 - protein, 248 aa, Synthetic construct
ERLVESGGGVVQPGSSLRLSCAASGFDFSRQGMHWVRQAPGQGLEWVAFIKYDGSEKYHADSVWGRLSISRDNSKDTLYLQMNSLRVEDTATYFCVREAGGPDYRNGYNYYDFYDGYYNYHYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGLEVLFQ

SEQ ID NO: 2 - protein, 216 aa, Synthetic construct
QSALTQPASVSGSPGQSITISCQGTSNDVGGYESVSWYQQHPGKAPKVVIYDVSKRPSGVSNRFSGSKSGNTASLTISGLQAEDEGDYYCKSLTSRSHRVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 28 aa, Synthetic construct
EAGGPDYRNGYNYYDFDDGYYNYHYMDV

SEQ ID NO: 4 - protein, 9 aa, Synthetic construct
FDFSRQGMH

SEQ ID NO: 5 - protein, 17 aa, Synthetic construct
FIKYDGSEKYHADSVWG

SEQ ID NO: 6 - protein, 14 aa, Synthetic construct
QGTSNDVGGYESVS

SEQ ID NO: 7 - protein, 7 aa, Synthetic construct
DVSKRPS

SEQ ID NO: 8 - protein, 10 aa, Synthetic construct
KSLTSRSHRV

SEQ ID NO: 9 - protein, 248 aa, Synthetic construct
ERLVESGGGVVQPGSSLRLSCAASGFDFSRQGMHWVRQAPGQGLEWVAFIKYDGSEKYHADSVWGRLSISRDNSKDTLYLQMNSLRVEDTATYFCVREAGGPDYRNGYNYYDFDDGYYNYHYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGLEVLFQ

SEQ ID NO: 10 - protein, 216 aa, Synthetic construct
QSALTQPASVSGSPGQSITISCQGTSNDVGGYESVSWYQQHPGKAPKVVIYDVSKRPSGVSNRFSGSKSGNTASLTISGLQAEDEGDYYCKSLTSRSHRVFGTGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 11 - protein, 28 aa, Synthetic construct
EAGGPDYRNGYYYYDFYDGYYNYHYMDV

SEQ ID NO: 12 - protein, 9 aa, Synthetic construct
KSLTSRSHR

SEQ ID NO: 13 - protein, 248 aa, Synthetic construct
ERLVESGGGVVQPGSSLRLSCAASGFDFSRQGMHWVRQAPGQGLEWVAFIKYDGSEKYHADSVWGRLSISRDNSKDTLYLQMNSLRVEDTATYFCVREAGGPDYRNGYYYYDFYDGYYNYHYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGLEVLFQ

SEQ ID NO: 14 - protein, 252 aa, Synthetic construct
QVQLVQSGAEVKKPGSSVKVSCKASGNSFSNHDVHWVRQATGQGLEWMGWMSHEGDKTGLAQKFQGRVTITRDSGASTVYMELRGLTADDTAIYYCLTGSKHRLRDYFLYNEYGPNYEEWGDYLATLDVWGHGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGLEVLFQ

SEQ ID NO: 15 - protein, 219 aa, Synthetic construct
EVVITQSPLFLPVTPGEAASLSCKCSHSLQHSTGANYLAWYLQRPGQTPRLLIHLATHRASGVPDRFSGSGSGTDFTLKISRVESDDVGTYYCMQGLHSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 31 aa, Synthetic construct
GSKHRLRDYFLYNEYGPDYEEWGDYLATLDV

SEQ ID NO: 17 - protein, 9 aa, Synthetic construct
NSFSNHDVH

SEQ ID NO: 18 - protein, 16 aa, Synthetic construct
MSHEGDKTGLAQKFQG

SEQ ID NO: 19 - protein, 14 aa, Synthetic construct
SHSLQHSTGANYLA

SEQ ID NO: 20 - protein, 7 aa, Synthetic construct
LATHRAS

SEQ ID NO: 21 - protein, 9 aa, Synthetic construct
MQGLHSPWT

SEQ ID NO: 22 - protein, 252 aa, Synthetic construct
QVQLVQSGAEVKKPGSSVKVSCKASGNSFSNHDVHWVRQATGQGLEWMGWMSHEGDKTGLAQKFQGRVTITRDSGASTVYMELRGLTADDTAIYYCLTGSKHRLRDYFLYNEYGPDYEEWGDYLATLDVWGHGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGLEVLFQ

(19) United States
(12) Patent Application Publication
(10) Pub. No.: US 2026/0152546 A1
(43) Pub. Date: Jun. 4, 2026

(54) NOVEL ANTIBODIES FOR HIV AND METHODS OF MAKING AND USING SAME

(71) Applicants: Duke University, Durham, NC (US); UNITED STATES OF AMERICA, AS REPRESENTED BY THE SECRETARY OF HEALTH AND HUMAN SERVICES, Washington, DC (US)

(72) Inventors: Bruce R. Donald, Durham, NC (US); Siyu Wang, Durham, NC (US); Anna U. Lowegard, Durham, NC (US); Graham T. Holt, Durham, NC (US); Marcel S. Frenkel, Durham, NC (US); Peter Kwong, Washington, DC (US); Jason Gorman, Washington, DC (US); Nicole Doria-Rose, Washington, DC (US); Gwo Yu Chuang, Washington, DC (US)

(21) Appl. No.: 19/126,308

(22) PCT Filed: Nov. 3, 2023
(86) PCT No.: PCT/US2023/078631

§ 371 Date: May 1, 2025

Related U.S. Application Data
(60) Provisional application No. 63/422,250, filed on Nov. 3, 2022.

(51) Int. Cl. C07K 16/114; A61P 31/18

(52) U.S. Cl. CPC C07K 16/1145; A61P 31/18; C07K 2317/76; C07K 2317/92

(57) ABSTRACT

Provided herein are potent broadly neutralizing antibodies (bNAbs) against HIV-1, and methods of using the same.

### STATEMENT REGARDING RELATED APPLICATIONS

This application is a national phase application under 35 U.S.C. § 371 of PCT International Application No.: PCT/US2023/078631, filed on Nov. 3, 2023, which claims the benefit of U.S. Provisional Patent Application No. 63/422,250, filed Nov. 3, 2022, the entire contents of which are incorporated herein by reference for all purposes.

### STATEMENT OF GOVERNMENT INTEREST

This invention was made with government support under GM144042, GM078031, and GM118543 awarded by the National Institutes of Health. The government has certain rights in the invention.

### SEQUENCE LISTING

The text of the computer readable sequence listing filed herewith, titled “DUKE_42359_601_SequenceListing”, created Nov. 2, 2023, having a file size of 21,992 bytes, is hereby incorporated by reference in its entirety.

### BACKGROUND

Broad and potent antibodies against HIV-1 show therapeutic promise for preventing viral transmission or infection and have been shown to suppress viremia in humans. The HIV-1 envelope (Env) apex, comprised of variable loops V1 and V2, is a potential target site for anti-HIV-1 broadly neutralizing antibodies (bNAbs) despite the high antigen sequence variation at the V1V2 region and the presence of a protective glycan shield. These bNAbs form a category that contains the PG9 and PGT145 antibody classes. Their utility as therapeutics or for prevention, however, are impaired by their potency and breadth. Accordingly, what is needed are methods for increasing potency and breadth of HIV antibodies, including bNAbs.

### SUMMARY

In some aspects, provided herein are anti-HIV-1 broadly neutralizing antibodies (bNAbs). In some embodiments, provided herein are anti-HIV-1 broadly neutralizing antibodies comprising a heavy chain comprising a sequence having at least 80% sequence identity to SEQ ID NO: 1, wherein the heavy chain comprises a Y to D substitution mutation at position 114 relative to SEQ ID NO: 1, and a light chain having at least 80% sequence identity to SEQ ID NO: 2. In some embodiments, the heavy chain comprises a sequence having at least 90% sequence identity to SEQ ID NO: 1 and the light chain comprises a sequence having at least 90% sequence identity to SEQ ID NO: 2. In some embodiments, the heavy chain comprises a sequence having at least 95% sequence identity to SEQ ID NO: 1 and the light chain comprises a sequence having at least 95% sequence identity to SEQ ID NO: 2. In some embodiments, the heavy chain comprises a complementary determining region 3 (CDRH3) having a sequence of SEQ ID NO: 3. In some embodiments, the heavy chain comprises complementary determining regions (CDRs) CDRH1, CDRH2, and CDRH3 comprising SEQ ID NO: 4, SEQ ID NO: 5, and SEQ ID NO: 3, respectively. In some embodiments, the light chain comprises CDRS CDRL1, CDRL2, and CDRL3 comprising SEQ ID NO: 6, SEQ ID NO: 7, and SEQ ID NO: 8, respectively. In some embodiments, the heavy chain comprises a sequence of SEQ ID NO: 9 and the light chain comprises a sequence of SEQ ID NO: 2.

In some embodiments, provided herein are anti-HIV-1 broadly neutralizing antibodies comprising a heavy chain comprising a sequence having at least 80% sequence identity to SEQ ID NO: 1, wherein the heavy chain comprises a N to Y substitution mutation at position 109 relative to SEQ ID NO: 1, and a light chain having at least 80% sequence identity to SEQ ID NO: 10. In some embodiments, the heavy chain comprises a sequence having at least 90% sequence identity to SEQ ID NO: 1 and the light chain comprises a sequence having at least 90% sequence identity to SEQ ID NO: 10. In some embodiments, the heavy chain comprises a sequence having at least 95% sequence identity to SEQ ID NO: 1 and the light chain comprises a sequence having at least 95% sequence identity to SEQ ID NO: 10. In some embodiments, the heavy chain comprises a complementary determining region 3 (CDRH3) having a sequence of SEQ ID NO: 11. In some embodiments, the heavy chain comprises complementary determining regions (CDRs) CDRH1, CDRH2, and CDRH3 comprising SEQ ID NO: 4, SEQ ID NO: 5, and SEQ ID NO: 11, respectively. In some embodiments, the light chain comprises CDRS CDRL1, CDRL2, and CDRL3 comprising SEQ ID NO: 6, SEQ ID NO: 7, and SEQ ID NO: 12, respectively. In some embodiments, the heavy chain comprises a sequence of SEQ ID NO: 13 and wherein the light chain comprises a sequence of SEQ ID NO: 10.

In some embodiments, provided herein are anti-HIV-1 broadly neutralizing antibodies comprising a heavy chain comprising a sequence having at least 80% sequence identity to SEQ ID NO: 14, wherein the heavy chain comprises a N to D substitution mutation at position 116 relative to SEQ ID NO: 14, and a light chain having at least 80% sequence identity to SEQ ID NO: 15. In some embodiments, the heavy chain comprises a sequence having at least 90% sequence identity to SEQ ID NO: 14 and the light chain comprises a sequence having at least 90% sequence identity to SEQ ID NO: 15. In some embodiments, the heavy chain comprises a sequence having at least 95% sequence identity to SEQ ID NO: 14 and the light chain comprises a sequence having at least 95% sequence identity to SEQ ID NO: 15. In some embodiments, the heavy chain comprises a complementary determining region 3 having a sequence of SEQ ID NO: 16. In some embodiments, the heavy chain comprises complementary determining regions (CDRs) CDRH1, CDRH2, and CDRH3 comprising SEQ ID NO: 17, SEQ ID NO: 18, and SEQ ID NO: 16, respectively. In some embodiments, the light chain comprises CDRS CDRL1, CDRL2, and CDRL3 comprising SEQ ID NO: 19, SEQ ID NO: 20, and SEQ ID NO: 21, respectively. In some embodiments, the heavy chain comprises a sequence of SEQ ID NO: 22 and wherein the light chain comprises a sequence of SEQ ID NO: 15.

The antibodies provided herein find use in methods of treating viral infection in a subject. In some embodiments, provided herein are methods of treating viral infection in a subject, comprising providing to a subject having or suspected of having a viral infection an antibody provided herein. In some embodiments, the viral infection is an HIV infection.

### BRIEF DESCRIPTION OF THE DRAWINGS

FIGS. 1A-1G neutralization breadth and potency for PGT145 and PG9RSH variants assayed on a panel of 208 pseudoviruses. FIG. 1A and FIG. 1B show breadth/potency curves for PGT145 and PG9RSH variants and controls, respectively. Curves represent the fraction of pseudoviruses that were neutralized with IC₅₀ smaller than the given cutoff. An increase in breadth and potency is indicated by a shift upward and left. PGT145 variant DU303 and PG9RSH variants N(100f)Y and DU025 improve breadth and potency relative to wild type. For IC₈₀ curves, see FIG. S3. FIG. 1C shows breadth/potency curves for PG9RSH DU025 and PGDM1400. Despite slightly weaker median and mean neutralization potency, the overall breadth and potency of DU025 rival that of the PGDM1400 antibody. FIGS. 1D, 1E, and 1F show neutralization dendrograms for variants DU303, PG9RSH N(100f)Y, and DU025, respectively. Pseudoviruses are grouped into clades by sequence similarity, forming a tree graph. Internal branches in the tree denote groups of viruses. Terminal branches, corresponding to a single pseudovirus, are colored by IC₅₀. FIG. 1G is a summary of large-panel neutralization breadth and potency for variants and controls, measured by IC₈₀.

FIGS. 2A-2C show Cryo-EM structures of PG9RSH and PGT145 variants in complex with BG505 DS-SOSIP Env trimer. Backbone shown in ribbon representation with glycans, and amino acids shown as sticks or lines. Env subunits and antibodies are shown. Distances (Å) are shown with dotted lines, and energetic interactions are shown with Probe dots. Members of the PG9RSH (A and B) and PGT145 lineages (C) interact with the trimer apex and are characterized by a negatively charged CDRH3 that is hammer-like or extended, respectively. FIG. 2A shows that the PGT145 mutation N(100l)D forms more favorable electrostatic interactions with gp120. FIG. 2B shows that the PG9RSH N(100f)Y mutation (variant DU011) creates interactions with a side-chain nitrogen from gp120 residue K168, forming geometry consistent with a π-cation interaction. FIG. 2C shows that the PG9RSH Y(100k)D mutation forms long-range interactions with polar and positively charged residues Q170 and K305.

FIGS. 3A-3F show that the OSPREY design ensembles correctly predicted structural features for PG9RSH and PGT145 variants. Ten members of the low-energy ensemble (LEE) predicted by OSPREY are shown for variants of PGT145 (FIG. 3A) and PG9RSH (FIG. 3B and FIG. 3C) above corresponding cryo-EM structures (FIG. 3D-FIG. 3F). Backbones are shown as ribbons with amino acids shown as lines or as sticks. Distances (A) are shown with dotted lines. FIG. 3A shows that the PGT145 mutation N(100l)D is predicted to form electrostatic interactions with gp120 residues R166 and K169. A carboxyl oxygen of D(100l) lies 5 and 4.2 Å from side-chain nitrogens of gp120 residues R166 and K169, respectively. Despite a lateral translation of the CDRH3 loop relative to the trimer apex, the LEE correctly predicts features of the experimental structure shown in FIG. 3D. FIG. 3B shows the PG9RSH N(100f)Y mutation creates interactions with gp120 residue K168. The side-chain amino nitrogen of K168 lies 5.1 Å from the ring plane of Y(100f), forming geometry consistent with a π-cation interaction. The LEE correctly predicts interactions found in the experimentally determined structure shown in FIG. 3E. FIG. 3C shows the PG9RSH Y(100k)D mutation is predicted to form electrostatic interactions with polar and charged residues on gp120. A carboxyl oxygen of D(100k) lies 3.2 Å from the side-chain nitrogen of Q170 and 5.6 Å from R308. The LEE correctly predicts interactions with Q170, but a translation and rotation of the CDRH3 loop places R308 further away as shown in FIG. 3F.

FIG. 4 shows K* scores for a double-mutation design of PGT145 at residues 100d and 100l. Bounds on the K* score for single mutations predicted using OSPREY are shown as horizontal bars. Pictured results are limited to the top 50 design predictions. Designs for which the unbound antibody is predicted to be more stable, approximately equally-stable, or less stable than wild-type are indicated by coloring. Relative stability was estimated using the lower-bound on the partition-function value for the unbound antibody state. Wild-type antibody residue labeled in bold. The bounds on the K* score for the wild-type design are indicated by the gray shaded box.

FIG. 5A and FIG. 5B show selected design results for PG9RSH. Bounds on the K* score for single mutations predicted using OSPREY are shown as horizontal bars. Designs for which the unbound antibody is predicted to be more stable, approximately equally-stable, or less stable than wild-type are indicated by coloring. Relative stability was estimated using the lower-bound on the partition-function value for the unbound antibody state. Wild-type antibody residue labeled in bold. The bounds on the K* score for the wild-type design are indicated by the gray shaded box.

FIGS. 6A-6C show that variant neutralization improves over wild-type measured by IC80. FIG. 6A and FIG. 6B show breadth/potency curves for PGT145 and PG9RSH variants and controls, respectively. Curves represent the fraction of pseudoviruses that were neutralized with IC80 smaller than the given cutoff. An increase in breadth and potency is indicated by a shift upward and left. The variants PGT145 N(100l)D (DU303), PG9RSH N(100f)Y (DU011) and PG9RSH Y(100k)D (DU025) improve breadth and potency relative to wild-type. FIG. 6C shows breadth/potency curves for PG9RSH DU025 and PGDM1400. Despite slightly weaker median and mean neutralization potency, DU025 exhibits comparable breadth and potency to the PGDM1400 antibody.

FIG. 7 shows fold change in neutralization for PGT145 and PG9RSH variants. A histogram of fold decrease in IC50 values for each antibody/pseudovirus pair is shown. Pairs where IC50 exceeded 50 μg/ml are not shown. The dotted vertical line indicates a fold increase of 1, or not change. All variants exhibited shifts to the right, indicating a trend of increased potency and breadth. This improvement is most marked in PGT145 DU303.

FIGS. 8A-8C show difference dendograms demonstrating patterns of neutralizing relative to wild-type. The differences in neutralization (fold-decrease in IC50) between each antibody and its wild-type ancestor are shown using difference dendograms for all large-panel viruses for variants DU303 (FIG. 8A), DU011 (FIG. 8B), and DU025 (FIG. 8C). Pseudoviruses are grouped into clades by sequence similarity, forming a tree graph. Internal branches in the tree denote groups of viruses. Terminal branches correspond to single pseudovirsues, and are colored by the fold-decrease in IC₅₀ relative to the wildtype antibody, whereas fold changes in neutralization potency are indicated by a color gradient. As shown in FIG. 10A, DU303 exhibits increased neutralization for many viruses in lades C/BC, AG, A/ACD/AD, AE, and D/CD, but shows decreases in neutralization for viruses in clade B. AS shown in FIG. 10B, DU011 increases virus neutralization for almost all clades, with the possible exception of clade A/ACD/AD. As shown in FIG. 11C. DU025 exhibits increased neutralization for some viruses in all clades.

FIG. 9 shows fold decrease in IC50 over wild-type by pseudovirus. Data from large neutralization panel excluding pseudoviruses for which antibody variant or wild-type IC50 was less than 50 μg/ml. Dotted line indicates a fold change of 1 (e.g. no change). Horizontal black lines indicate the geometric mean for each variant.

FIGS. 10A-10C are histograms showing fold decrease in IC50 by clade. The dotted vertical line indicates no change. A rightward distribution shift indicates improvement from wildtype.

FIGS. 11A-11C show ROC curves for repeated, nested cross-validation of models. Models were evaluated using (5-times) repeated, 10-fold nested cross-validation. ROC curves are shown as light traces. The mean ROC curve over all outer cross-validation steps is shown as a black rack, with shaded areas represented +/−1 standard deviation. Model for DU303 is shown in FIG. 11A, DU011 is shown in FIG. 11B, and DU025 is shown in FIG. 11C. Models for DU303 (FIG. 11A) give good predictive power on average.

FIG. 12 shows variable importances for the DU303 predictive model. Env residues K169 and R169 predict change in neutralization between PGT145 and DU303, indicated by two separate variable importance measures. FIG. 12, left panel shows residues ranked by mean decrease in impurity (MDI). FIG. 12, right panel shows the permutation importance which measures the change in loss function created by randomly permuting a feature.

FIG. 13 shows distribution of changes in viral neutralization highlighting the importance of ENV residue 169 for neutralization by DU303. No change in neutralization is indicated with a dashed vertical line. AS shown in FIG. 12A, the distribution over all viruses with neutralization less than 50 μg/mL is shifted to the right, indicating that, in general, viruses are neutralized more potently by the DU303 variant than the wildtype PGT145. As shown in FIG. 12B, distributions over viruses with Env proteins that contain a positively-charged residue at position 169 or viruses without a positively charged residue at this position are well-separated. This indicates the importance of the amino acid at this position to the neutralization by DU303.

FIG. 14 shows sequence information entropy of variable loops in large panel. The number of bits of entropy for each residue in the V1, V2, and V3 loops (excluding hypervariable regions) of HIV Env in the large neutralization panel is shown in FIG. 14A, FIG. 14B, and FIG. 14C, respectively. Residue numbering and entropy calculations are based on alignment to the HXB2 reference sequence. Residues predicted to be important are colored according to the legend.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### 1. Definitions

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having.” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient).

Unless otherwise defined, all technical terms used herein have the same meaning as commonly understood by one of ordinary skill in the art to which this disclosure belongs.

Each of the various references, presentations, publications, provisional and/or non-provisional U.S. patent applications, U.S. patents, non-U.S. patent applications, and/or non-U.S. patents that have been identified herein, is incorporated herein in its entirety by this reference.

### 2. Antibodies

Extensive structural characterization of bNAb lineages has suggested that breadth of neutralization is conferred by favorable interactions with conserved epitope features. Both the eponymous PG9 (McLellan et al., Nature, 480 (2011), pp. 336-343; Walker et al., Science, 326 (2009), pp. 285-289) and PGT145 (Walker et al., Nature, 477 (2011), pp. 466-470; Lee et al., Immunity, 46 (2017), pp. 690-702; Liu et al., Nat. Struct. Mol. Biol., 24 (2017), pp. 370-378) monoclonal antibodies achieve neutralization breadth by targeting conserved structural features on the Env apex. PG9 uses its long, axe-like CDRH3 loop to form hydrogen bonds with the C strand of the Env V2 region in a parallel beta-strand conformation and also to interact productively with several apex glycans, including those at Env residues N160, N156, and, in some cases, N173. The beta-strand interaction allows PG9 to maintain favorable contacts with the V2 region despite variation in Env side-chain identities. PG9 was previously modified by incorporating mutations from the PG16 antibody, yielding the antibody PG9-16-RSH (herein, PG9RSH) (Nat. Struct. Mol. Biol., 20 (2013), pp. 804-813). PGT145 uses its long, needle-like CDRH3 loop to insert sulfated tyrosines into the Env apex hole to contact sites of conserved positive charge, both on the C strand and deeper beneath the surface of the Env trimer. These strategies contrast with those used by members of the VRC38 class of antibodies (a member of the same V1V2 bNAb category), which rely on specific V2 C-strand side-chain interactions to neutralize HIV and, perhaps as a result, exhibit much narrower breadth of neutralization.

The relationship between breadth and potency of neutralization is of considerable interest for antibody design. Although some studies have indicated that improving neutralization against a single antigen can lead to improved neutralization breadth, other evidence suggests the existence of a trade-off between breadth and potency. Exploration of this relationship from a structural perspective is made more challenging by the relative scarcity of high-resolution structure information compared with the extreme antigenic diversity of targets like HIV.

Herein, the PG9RSH and PGT145 anti-HIV-1 bNAbs were designed for improved potency and breadth using the computational protein design software OSPREY (J. Comput. Chem., 39 (2018), pp. 2494-2507). The bNAbs designed and tested herein are described in Holt et al, Cell Reports (2023) 42; 7, 112711. Predicted potency for the BG505 strain was used as a proxy for predicted neutralization breadth, and interactions with both conserved and non-conserved epitope residues were computationally optimized. Three bNAb single-mutation variants are presented and characterized herein. Specifically, the variants DU025, DU303, and DU011 were generated and compared to their respective wild-type (PG9RSH and PGT145) and PGDM1400 antibodies. Measured improvements in breadth or potency relative to wild type were observed. Cryoelectron microscopy (cryo-EM) structures for these three designed variants were determined to provide atomic-level insight into increases in breadth and potency. The largest improvements in median potency (≈3-fold IC₅₀, ≈4-fold IC₈₀) occurred for PG9RSH variant DU025, which achieves neutralization breadth and potency rivaling that of the antibody PGDM1400. Surprisingly, the largest improvements in breadth occurred for a variant that optimizes interactions with variable epitope residues. This variant, PGT145 DU303, lost subtype potency for clade B but nonetheless improved overall breadth of neutralization from 39% (wild type) to 54% at clinically relevant concentrations (IC₈₀<1 μg/mL). For this designed antibody, increases in potency of >100-fold for six pseudoviruses across five clades was observed. Moreover, the median improvement in IC₈₀ (across 208 strains) was over 3-fold.

In some aspects, provided herein are anti-HIV-1 broadly neutralizing antibodies. As used herein, the term “broadly neutralizing antibody” or “bNAb” refers to an antibody that neutralizes a wide variety of HIV strains. There are two main types of human immunodeficiency virus (HIV), HIV-1 and HIV-2. HIV-1 is the most common type of HIV, whereas HIV-2 occurs in a much smaller population of people mostly in West Africa. Both HIV-1 and HIV-2 have multiple groups, and each group has multiple subtypes, also referred to as strains or clades. For example, HIV-1 group M (major) has nine identified strains so far, A, B, C, D, F, G, H, J, and K. The term bNAb indicates an antibody with neutralizing activity against multiple HIV strains.

In some embodiments, provided herein is an anti-HIV-1 broadly neutralizing antibody (bNAb) comprising a heavy chain comprising a sequence having at least 80% sequence identity (e.g. at least 80%, at least 85%, at least 90%, at least 91%, at least 92%, at least 93%, at least 94%, at least 95%, at least 96%, at least 97%, at least 98%, at least 99% identity) to SEQ ID NO: 1, wherein the heavy chain comprises a Y to D substitution mutation at position 114 relative to SEQ ID NO: 1, and a light chain having at least 80% sequence identity (e.g. at least 80%, at least 85%, at least 90%, at least 91%, at least 92%, at least 93%, at least 94%, at least 95%, at least 96%, at least 97%, at least 98%, at least 99% identity) to SEQ ID NO: 2. In some embodiments, the heavy chain comprises a sequence having at least 90% sequence identity to SEQ ID NO: 1 and the light chain comprises a sequence having at least 90% sequence identity to SEQ ID NO: 2. In some embodiments, the heavy chain comprises a sequence having at least 95% sequence identity to SEQ ID NO: 1 and the light chain comprises a sequence having at least 95% sequence identity to SEQ ID NO: 2. In some embodiments, the heavy chain comprises a complementary determining region 3 having a sequence of SEQ ID NO: 3. In some embodiments, the heavy chain comprises a sequence of SEQ ID NO: 9. In some embodiments, the heavy chain comprises a sequence of SEQ ID NO: 9 the light chain comprises a sequence of SEQ ID NO: 2. An antibody comprising a heavy chain comprising the sequence of SEQ ID NO: 9 and a light chain comprising the sequence of SEQ ID NO: 2 is referred to herein as PG9RSH Y(100k)D, or DU025.

In some embodiments, provided herein is an anti-HIV-1 bNAb comprising a heavy chain comprising a sequence having at least 80% sequence identity (e.g. at least 80%, at least 85%, at least 90%, at least 91%, at least 92%, at least 93%, at least 94%, at least 95%, at least 96%, at least 97%, at least 98%, at least 99% identity) to SEQ ID NO: 1, wherein the heavy chain comprises a N to Y substitution mutation at position 109 relative to SEQ ID NO: 1, and a light chain having at least 80% sequence identity (e.g. at least 80%, at least 85%, at least 90%, at least 91%, at least 92%, at least 93%, at least 94%, at least 95%, at least 96%, at least 97%, at least 98%, at least 99% identity) to SEQ ID NO: 10. In some embodiments, the heavy chain comprises a sequence having at least 90% sequence identity to SEQ ID NO: 1 and the light chain comprises a sequence having at least 90% sequence identity to SEQ ID NO: 10. In some embodiments, the heavy chain comprises a sequence having at least 95% sequence identity to SEQ ID NO: 1 and the light chain comprises a sequence having at least 95% sequence identity to SEQ ID NO: 10. In some embodiments, the heavy chain comprises a complementary determining region 3 having a sequence of SEQ ID NO: 11. In some embodiments, the heavy chain comprises a sequence of SEQ ID NO: 13. In some embodiments, the heavy chain comprises a sequence of SEQ ID NO: 13 the light chain comprises a sequence of SEQ ID NO: 10. An antibody comprising a heavy chain comprising the sequence of SEQ ID NO: 13 and a light chain comprising the sequence of SEQ ID NO: 10 is referred to herein as PG9RSH N(100f)Y or DU011.

In some embodiments, provided herein is an anti-HIV-bNAb comprising a heavy chain comprising a sequence having at least 80% sequence identity e.g. at least 80%, at least 85%, at least 90%, at least 91%, at least 92%, at least 93%, at least 94%, at least 95%, at least 96%, at least 97%, at least 98%, at least 99% identity) to SEQ ID NO: 14, wherein the heavy chain comprises a N to D substitution mutation at position 116 relative to SEQ ID NO: 14, and a light chain having at least 80% sequence identity e.g. at least 80%, at least 85%, at least 90%, at least 91%, at least 92%, at least 93%, at least 94%, at least 95%, at least 96%, at least 97%, at least 98%, at least 99% identity) to SEQ ID NO: 15. In some embodiments, the heavy chain comprises a sequence having at least 90% sequence identity to SEQ ID NO: 14 and the light chain comprises a sequence having at least 90% sequence identity to SEQ ID NO: 15. In some embodiments, the heavy chain comprises a sequence having at least 95% sequence identity to SEQ ID NO: 14 and the light chain comprises a sequence having at least 95% sequence identity to SEQ ID NO: 15. In some embodiments, the heavy chain comprises a complementary determining region 3 having a sequence of SEQ ID NO: 16. In some embodiments, the heavy chain comprises a sequence of SEQ ID NO: 22 and the light chain comprises a sequence of SEQ ID NO: 15. An antibody comprising a heavy chain comprising the sequence of SEQ ID NO: 22 and a light chain comprising the sequence of SEQ ID NO: 15 is referred to herein as PGT145 N(100l)D or DU0303.

The antibodies provided herein have improved breadth and/or potency compared to wildtype. For example, PG9RSH Y(100k)D (DU025) and PG9RSH N(100f)Y (DU011) have improved breadth and/or potency compared to the antibody PG9RSH. As another example, PGT145 N(100l)D (DU0303) has improved breath and/or potency compared to the antibody PGT145.

The antibodies provided herein find use in methods of treating viral infection in a subject. In some embodiments, the viral infection is an HIV infection. In some embodiments, the HIV infection is with HIV-1.

In some aspects, provided herein are methods of treating a viral infection in a subject, comprising providing to the subject an antibody provided herein. For example, in some embodiments the methods comprise providing to the subject an antibody provided herein for treatment of HIV infection in the subject. In some embodiments, the subject is diagnosed with or at risk of having infection with HIV-1.

The antibody may be provided to the subject by any suitable route, including parenteral routes (e.g. oral or by injection, such as intravenous, intramuscular, subcutaneous, intraarterial, etc.). Any suitable dose of the antibody may be provided to the subject at any suitable dosing interval to achieve the desired result. For example, in some embodiments the antibody is administered to the subject multiple times per day, daily, every 2 days, every 3 days, every 4 days, every 5 days, every 6 days, once per week, once every two weeks, once every 3 weeks monthly, every 2 months, every 3 months, every 4 months, every 5 months, every 6 months, or less than once every 6 months.

The following Examples are provided by way of illustration and not by way of limitation.

### EXAMPLES

### Example 1

Herein PG9RSH and PGT145 anti-HIV-1 bNAbs were designed for improved potency and breadth using the computational protein design software OSPREY (J. Comput. Chem., 39 (2018), pp. 2494-2507, 10.1002/jcc.25522). Potency for the BG505 strain was predicted as a proxy for predicted neutralization breadth, and interactions with both conserved and non-conserved epitope residues were computationally optimized. Three bNAb single-mutation variants are presented and characterized herein. These three bNAb single-mutation variants were compared to both wild-type (PG9RSH and PGT145) and PGDM1400 antibodies, and measured improvements in breadth or potency relative to wild type were observed. Cryoelectron microscopy (cryo-EM) structures for these three designed variants were observed to provide atomic-level insight into increases in breadth and potency. The largest improvements in median potency (≈3-fold IC₅₀, ≈4-fold IC₈₀) occurred for PG9RSH variant DU025, which achieves neutralization breadth and potency rivaling that of the antibody PGDM1400. Surprisingly, the largest improvements in breadth occurred for a variant that optimizes interactions with variable epitope residues. This variant, PGT145 DU303, lost subtype potency for clade B but nonetheless improved overall breadth of neutralization from 39% (wild type) to 54% at clinically relevant concentrations (IC₈₀<1 μg/mL). For this designed antibody, increases in potency of >100-fold were observed for six pseudoviruses across five clades. Moreover, the median improvement in IC₈₀ (across 208 strains) was over 3-fold.

### Results

OSPREY Predicts Mutations with Improved Affinity for PGT145 and PG9RSH

OSPREY was used to design variants of the PGT145 and PG9 antibodies using structures of each antibody bound to the BG505 SOSIP Env trimer (Protein Data Bank 5U1F, Protein Data Bank 5VJ6, rcsb.org). The effect of antibody mutations on antigen-binding affinity was predicted by computing K* scores (J. Comput. Biol., 12 (2005), pp. 740-761, 10.1089/cmb.2005.12.740) for both wild-type and mutant antibodies at 9 positions (Table 1) pruning or computing scores for approximately 14,000 sequences. An increase in score relative to wild type predicts an increase in Ka, i.e., improved binding. In general, antibody binding affinity for the Env spike correlates well with neutralization potency. These designs predicted that PGT145 N(100l)D (variant DU303), PG9RSH N(100f)Y, and PG9RSH Y(100k)D (variant DU025), among other substitutions, would improve the neutralization potency of PGT145 and PG9RSH.

TABLE 1
Flexible residues for designs of PG9RSH and PGT145

| PDB | | | | |
|---|---|---|---|---|
| ID | Name | Target | Mutable res | Flexible res |
| 5VJ6 | PG9:Env | 100l | H1839 | E953, F1418, |
| | | | | H1835, H1841 |
| 5VJ6 | PG9:Env | 100i, 100j, 100k | H1836, | E955, E956, |
| | | | H1837, | E957, E958, |
| | | | H1838 | E1091, H1754, |
| | | | | H1776, H1842 |
| 5VJ6 | PG9:Env | 100, 100b, 100c | H1827, | D503, D505, |
| | | | H1829, | E945, H1834 |
| | | | H1830 | |
| 5VJ6 | PG9:Env | 100e | H1832 | E951, E952, |
| | | | | E954, F1403 |
| 5VJ6 | PG9:Env | 100f | H1833 | E952, E953, |
| | | | | H1828, H1835 |
| 5VJ6 | PG9:Env | 100k | H1838 | E955, E1089, |
| | | | | E1091, E1092, |
| | | | | H1837 |
| 5U1F | PGT145:Env | 100l | H230 | A14, D146, |
| | | | | H222, H224, |
| | | | | H232 |
| 5U1F | PGT145:Env | 100d, 100l | H222, H230 | A15, D145, |
| | | | | D146, H219, |
| | | | | H232 |
| 5U1F | PGT145:Env | 100e, 100m | H223, H231 | C84, C87, D143, |
| | | | | H225 |

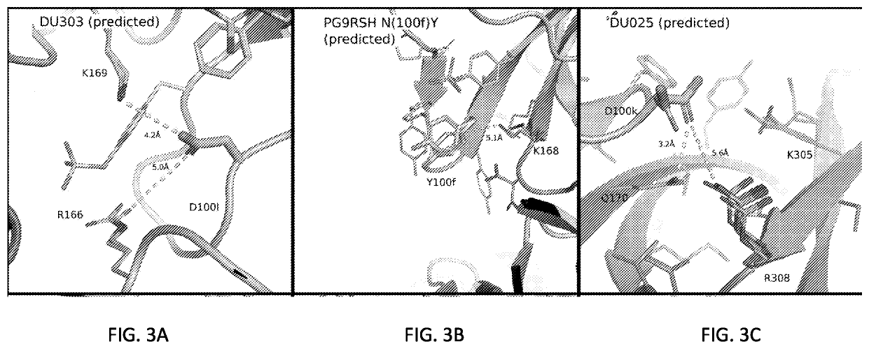

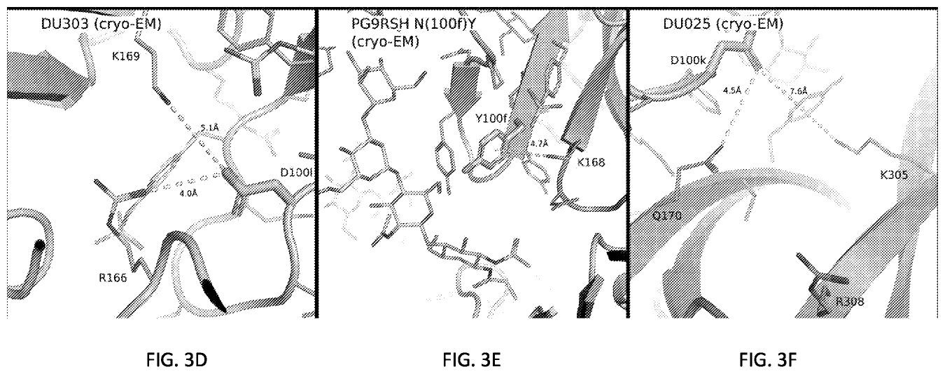

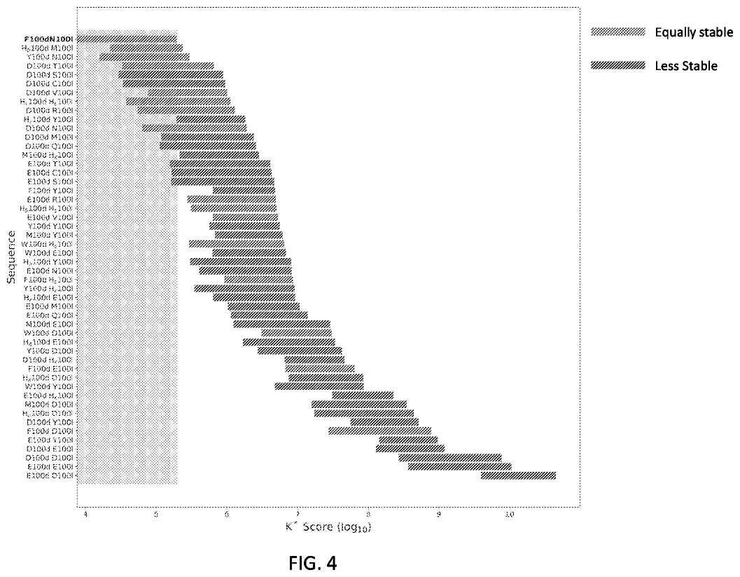

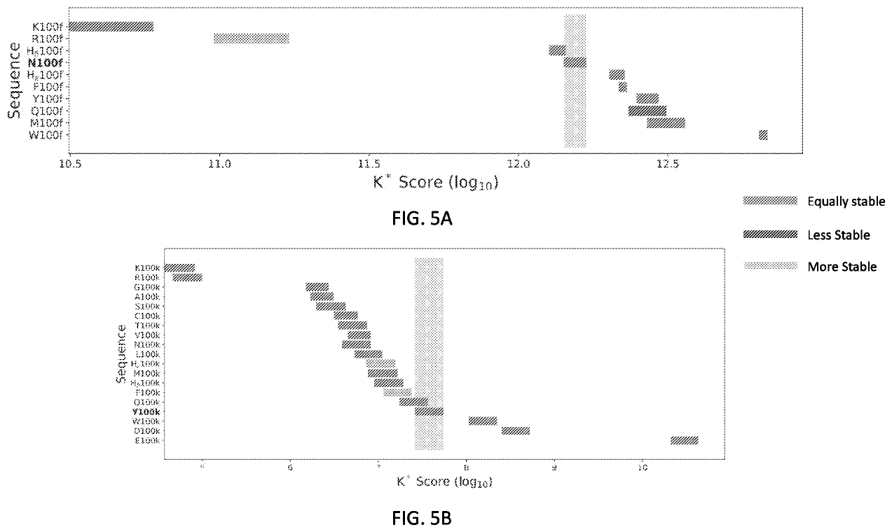

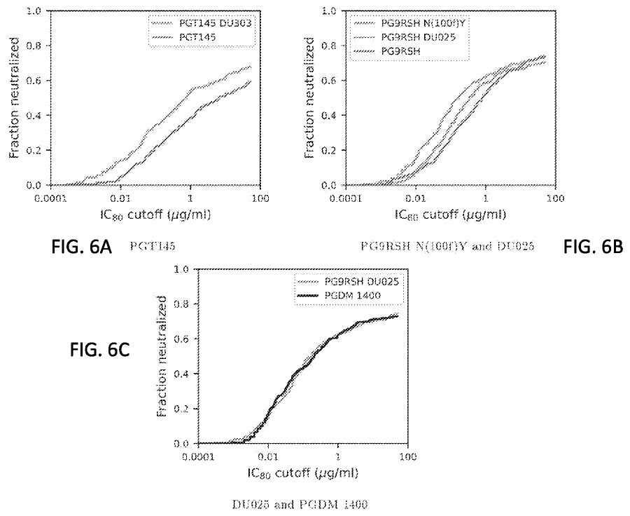

Double-mutation designs of PGT145 at residues F(100d) and N(100l) predicted that negatively charged substitutions at position 100l would improve binding affinity (FIG. 4) To avoid destabilization of the antibody CDRH3 conformation, sequences for which the lower bound on the partition function for the antibody state (ZAb) was less than 1017.7 were excluded from consideration. Mutations N(100l)D, F(100d)H/N(100l)D, and N(100l)E were all predicted to increase the K* score. Notably, these substitutions place a negatively charged side chain at residue 100l, perhaps leveraging proximity to the (generally) positively charged Env residues 166 and 169 (see FIG. 3C). A single-residue design of PG9RSH at residue N(100f) predicted that substitutions to Trp, Met, Tyr, His, or Phe would improve binding affinity (FIG. 5A; Table 2). An additional single-residue design of PG9RSH at residue Y(100k) predicted that large or negatively charged substitutions (Trp, Asp, or Glu) would improve binding affinity (FIG. 5B; Table 3).

TABLE 2
Upper and lower bounds on the K* score and partition
functions for PG9 designs at residue 100K predicted using OSPREY.
Hδ and Hε refer to the δ and ε protonation states of histidine.

| | log₁₀K* | | log₁₀ Z_{C} | | log₁₀ Z_{Ab} | | log₁₀ Z_{Env} | |
|---|---|---|---|---|---|---|---|---|
| 100f | UB | LB | UB | LB | UB | LB | UB | LB |
| W | 12.835 | 12.806 | 47.396 | 47.367 | 22.427 | 22.427 | 12.135 | 12.134 |
| M | 12.558 | 12.431 | 45.695 | 45.569 | 21.003 | 21.003 | 12.135 | 12.134 |
| Q | 12.495 | 12.370 | 44.582 | 44.458 | 19.953 | 19.953 | 12.135 | 12.134 |
| Y | 12.468 | 12.395 | 45.321 | 45.249 | 20.719 | 20.719 | 12.135 | 12.134 |
| F | 12.363 | 12.336 | 46.042 | 46.015 | 21.544 | 21.544 | 12.135 | 12.134 |
| H_{ε} | 12.355 | 12.303 | 45.848 | 45.798 | 21.359 | 21.359 | 12.135 | 12.134 |
| N¹ | 12.227 | 12.153 | 45.016 | 44.942 | 20.654 | 20.654 | 12.135 | 12.134 |
| H_{δ} | 12.159 | 12.101 | 45.910 | 45.853 | 21.617 | 21.617 | 12.135 | 12.134 |
| R | 11.232 | 10.980 | 45.929 | 45.686 | 22.571 | 22.563 | 12.135 | 12.134 |
| K | 10.778 | 10.496 | 45.163 | 44.884 | 22.254 | 22.250 | 12.135 | 12.134 |

¹Wild-type amino-acid.

TABLE 3
Upper and lower bounds on the K* score and partition
functions for PG9 designs at residue 100K predicted using OSPREY.
Hδ and Hε refer to the δ and ε protonation states of histidine.

| | log₁₀K* | | log₁₀ Z_{C} | | log₁₀ Z_{Ab} | | log₁₀ Z_{Env} | |
|---|---|---|---|---|---|---|---|---|
| 100k | UB | LB | UB | LB | UB | LB | UB | LB |
| E | 10.630 | 10.318 | 35.980 | 35.780 | 4.009 | 4.009 | 21.453 | 21.340 |
| D | 8.719 | 8.401 | 34.676 | 34.371 | 4.517 | 4.517 | 21.453 | 21.340 |
| W | 8.346 | 8.026 | 34.930 | 34.723 | 5.244 | 5.244 | 21.453 | 21.340 |
| Y¹ | 7.738 | 7.415 | 34.413 | 34.203 | 5.336 | 5.335 | 21.453 | 21.340 |
| Q | 7.563 | 7.238 | 33.259 | 33.048 | 4.357 | 4.356 | 21.453 | 21.340 |
| F | 7.372 | 7.057 | 34.360 | 34.159 | 5.649 | 5.649 | 21.453 | 21.340 |
| H_{δ} | 7.285 | 6.955 | 33.941 | 33.725 | 5.317 | 5.316 | 21.453 | 21.340 |
| M | 7.213 | 6.879 | 33.630 | 33.409 | 5.077 | 5.077 | 21.453 | 21.340 |
| H_{ε} | 7.189 | 6.861 | 33.952 | 33.738 | 5.424 | 5.424 | 21.453 | 21.340 |
| L | 7.043 | 6.733 | 33.330 | 33.133 | 4.947 | 4.947 | 21.453 | 21.340 |
| N | 6.915 | 6.588 | 33.342 | 33.128 | 5.087 | 5.087 | 21.453 | 21.340 |
| V | 6.914 | 6.655 | 29.480 | 29.334 | 1.226 | 1.226 | 21.453 | 21.340 |
| T | 6.876 | 6.544 | 33.197 | 32.978 | 4.981 | 4.981 | 21.453 | 21.340 |
| C | 6.760 | 6.494 | 32.636 | 32.483 | 4.536 | 4.536 | 21.453 | 21.340 |
| S | 6.627 | 6.293 | 32.978 | 32.758 | 5.012 | 5.012 | 21.453 | 21.340 |
| A | 6.489 | 6.231 | 32.849 | 32.704 | 5.020 | 5.020 | 21.453 | 21.340 |
| G | 6.436 | 6.178 | 32.446 | 32.301 | 4.670 | 4.670 | 21.453 | 21.340 |
| R | 5.000 | 4.663 | 30.997 | 30.773 | 4.657 | 4.657 | 21.453 | 21.340 |
| K | 4.911 | 4.575 | 30.690 | 30.467 | 4.440 | 4.440 | 21.453 | 21.340 |

¹Wild-type amino-acid.

### Neutralization Assessment Reveals Improvements in Breadth and Potency

Based on these designs, 10 and 34 variants of antibodies PGT145 and PG9RSH, respectively, were selected for small-panel neutralization assays (Table 4 and Table 5). These variants were selected by prioritizing variants with high K* scores and (to a lesser extent) high unbound-state partition functions (J. Comput. Biol., 25 (2018), pp. 1557-8666, 10.1089/cmb.2017.0267), and in some cases, promising mutations from different designs were combined. Variants DU303 (PGT145 N(100l)D), PG9RSH N(100f)Y, and DU025 (PG9RSH Y(100k)D) were selected for assay against a large panel of 208 pseudoviruses to further characterize their potency and breadth of neutralization. These variants were selected based on the number of pseudoviruses neutralized with an IC₈₀≤50 g/mL, the median IC₈₀ value, the number of pseudoviruses neutralized with an IC₅₀≤50 μg/mL, and the median IC₅₀ value (listed in order of importance). For example, variants N(100f)Y and DU017 were selected over DU014 because DU014 performs comparatively poorly as measured by IC₈₀. Although its IC₅₀ neutralization breadth appears to be greater, the additional neutralized virus is neutralized relatively poorly (IC₅₀=36.1 μg/mL) and disappears when measured using IC₈₀. Furthermore, the median IC₅₀ and IC₈₀ values of DU014 are greater than for N(100f)Y (less potent). Additionally, the sequence and neutralization diversity of the set of variants to be characterized was considered. DU303, DU025, and PG9RSH N(100f)Y (DU011) improved over wild-type activity in both breadth and potency of neutralization.

TABLE 4
PGT145 variants selected for small-panel neutralization assays

| Identifier | Variant Mutations |
|---|---|
| DU301 | F(100d)E |
| DU302 | L(100e)E |
| DU303 | N(100l)D |
| DU304 | Y(100m)E |
| DU305 | L(100e)E N(100l)D |
| DU306 | L(100e)E N(100l)D Y(100m)M |
| DU307 | F(100d)H L(100e)E N(100l)D |
| DU308 | F(100d)E L(100e)E N(100l)R |
| DU309 | F(100d)E L(100e)E N(100l)D |
| DU310 | F(100d)H L(100e)E N(100l)D Y(100m)H |

TABLE 5
PG9RSH variants selected for small-panel neutralization assays

| Identifier | Variant Mutations |
|---|---|
| DU001 | N(100c)H |
| DU002 | N(100c)K |
| DU003 | N(100c)M |
| DU004 | N(100c)R |
| DU005 | D100M R(100b)K N(100c)E |
| DU006 | D100W R(100b)K N(100c)E |
| DU007 | N(100f)K |
| DU008 | N(100f)R |
| DU009 | N(100f)W |
| DU010 | N(100j)H |
| DU011 | N(100f)Y |
| DU012 | N(100f)W Y(100k)W |
| DU013 | N(100f)Y Y(100k)W |
| DU014 | N(100c)W N(100f)Y |
| DU015 | N(100c)W N(100f)W |
| DU016 | N(100c)F N(100f)Y |
| DU017 | N(100c)F N(100f)W |
| DU018 | D100E N(100c)Y N(100f)W |
| DU019 | D100E N(100c)Y N(100f)Y |
| DU020 | D100E R(100b)K N(100c)W N(100f)W |
| DU021 | N(100f)M |
| DU022 | N(100f)Q |
| DU023 | F(100j)E |
| DU024 | Y(100k)E |
| DU025 | Y(100k)D |
| DU026 | N(100c)F N(100f)W F(100j)E |
| DU027 | N(100c)F N(100f)W Y(100k)E |
| DU028 | N(100c)F N(100f)W Y(100k)D |
| DU029 | N(100c)F Y(100e)D N(100f)W |
| DU030 | N(100c)F Y(100e)E N(100f)W |
| DU031 | N(100f)Y F(100j)E |
| DU032 | N(100f)Y Y(100k)E |
| DU033 | N(100f)Y Y(100k)D |
| DU034 | Y(100e)D N(100f)Y |

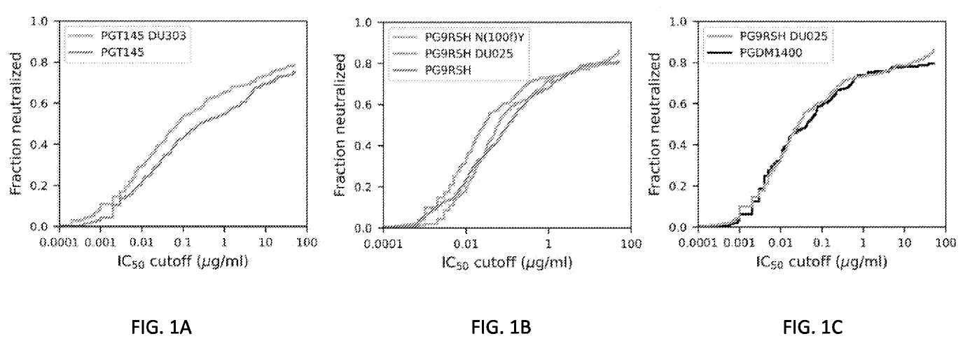

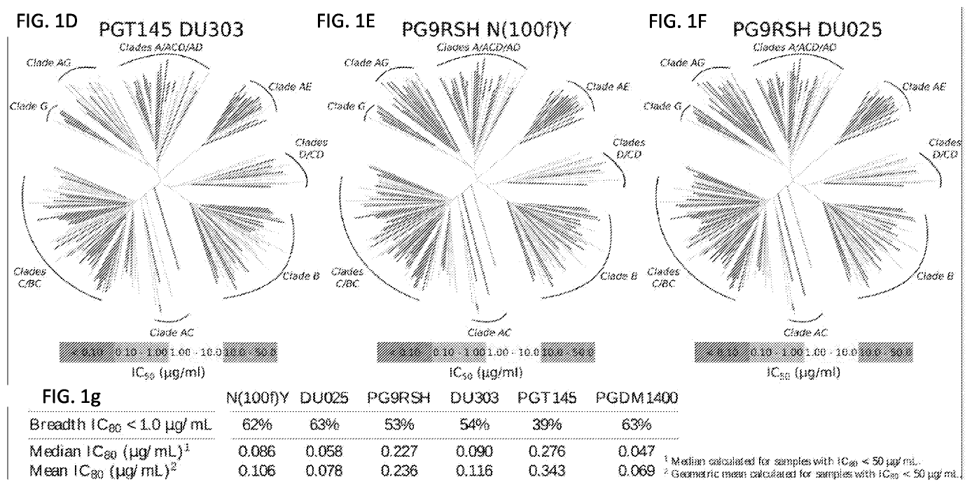

DU303 neutralized more pseudovirus strains with higher potency (FIG. 1A). DU303 increased neutralization potency against the BG505 strain by 3- and 25-fold as measured by IC₅₀ and IC₈₀, respectively: IC₅₀ decreased from 0.010 to 0.003 μg/mL, and IC₈₀ decreased from 0.253 to 0.010 μg/mL. Median neutralization potency across the large panel increased by 2- and 3-fold (IC₅₀ and IC₈₀): median IC₅₀ decreased from 0.053 to 0.024 μg/mL, and median IC₈₀ decreased from 0.276 to 0.090 μg/mL (Tables 5 and 6). DU303 improved neutralization breadth relative to PGT145: the percentage of tested pseudoviruses with measurable neutralization (IC₅₀<50 μg/mL) increased from 75% to 79% (Table 10). Interestingly, the improvement in breadth relative to PGT145 was more pronounced when evaluated at a cutoff with clinical relevance⁸: the percentage of viruses neutralized with IC₈₀<1 μg/mL increased from 39% to 54% (Table 6).

PG9RSH N(100f)Y increased median potency of neutralization but only slightly increased breadth (FIG. 1B). PG9RSH N(100f)Y increased neutralization potency against the BG505 strain by 2.8-fold as measured by IC₈₀ but showed no appreciable change in IC₅₀: IC₈₀ decreased from 0.065 to 0.023 μg/mL, and IC₅₀ remained at 0.009 μg/mL. Median neutralization potency across the large panel increased by 1.9- and 2.6-fold (IC₅₀ and IC₈₀): median IC₅₀ decreased from 0.047 to 0.025 μg/mL, and median IC₈₀ decreased from 0.227 to 0.086 μg/mL (Table 10 and Table 6). PG9RSH N(100f)Y slightly improved neutralization breadth relative to PG9RSH: the percentage of tested pseudoviruses with measurable neutralization (IC_{50<50} μg/mL) increased from 81% to 83% (Table 10). However, the improvement in breadth relative to PG9RSH was larger when evaluated for pseudoviruses neutralized with an IC_{80<1} μg/mL: breadth increased from 53% to 62% (Table 6).

DU025 increased potency and breadth of neutralization (FIG. 1B), and the resulting breadth-potency plot is qualitatively similar to that for PGDM1400 antibody (FIG. 1C). DU025 increased neutralization potency against the BG505 strain by 2.2- and 6.5-fold as measured by IC₅₀ and IC₈₀, respectively: IC₅₀ decreased from 0.009 to 0.004 μg/mL, and IC₈₀ decreased from 0.065 to 0.010 μg/mL. Median neutralization potency across the large panel increased by 2.7- and 3.9-fold (IC₅₀ and IC₈₀): median IC₅₀ decreased from 0.047 to 0.017 μg/mL, and median IC₈₀ decreased from 0.227 to 0.058 μg/mL (Tables 5 and 6). DU025 also improved neutralization breadth relative to PG9RSH: the percentage of tested pseudoviruses with measurable neutralization (IC₅₀<50 g/mL) increased from 81% to 87% (Table 10). Again, the improvement in breadth relative to PG9RSH was larger when evaluated at the clinically relevant cutoff of IC₈₀<1 μg/mL: breadth increased from 53% to 63% (Table 6). While the mean and median potencies for DU025 remained slightly weaker than for PGDM1400, overall, the breadth and potency of DU025 rivaled that of PGDM1400.

TABLE 10
Summary of large-panel neutralization results (IC50)

| | | N(100f)Y^{1, 3} | DU025 | PG9RSH | DU303 | PGT145 | PGDM1400 |
|---|---|---|---|---|---|---|---|
| #VS Assayed | | 208 | 208 | 208 | 208 | 208 | 208 |
| # | IC₅₀ 50 μg/mL | 173 | 181 | 169 | 164 | 157 | 167 |
| VS | IC₅₀ < 10 μg/mL | 165 | 165 | 168 | 153 | 145 | 164 |
| Neut. | IC₅₀ < 1.0 μg/mL | 156 | 153 | 143 | 137 | 114 | 155 |
| | IC₅₀ < 0.1 μg/mL | 125 | 128 | 104 | 113 | 91 | 124 |
| | IC₅₀ < 0.01 μg/mL | 52 | 66 | 48 | 61 | 43 | 68 |
| % | IC₅₀ 50 μg/mL | 83 | 87 | 81 | 79 | 75 | 80 |
| VS | IC₅₀ < 10 μg/mL | 79 | 79 | 81 | 74 | 70 | 79 |
| Neut. | IC₅₀ < 1.0 μg/mL | 75 | 74 | 69 | 66 | 55 | 75 |
| | IC₅₀ < 0.1 μg/mL | 60 | 62 | 50 | 54 | 44 | 60 |
| | IC₅₀ < 0.01 μg/mL | 25 | 32 | 23 | 29 | 21 | 33 |
| | Median IC₅₀ ² | 0.025 | 0.017 | 0.047 | 0.024 | 0.053 | 0.014 |
| | Mean IC₅₀¹ | 0.040 | 0.035 | 0.048 | 0.038 | 0.086 | 0.024 |

¹Geometric mean calculated for samples with IC₅₀ < 50 μg/mL.
² Median calculated for samples with IC₅₀ < 50 μg/mL.
³Variant of PG9RSH.

TABLE 6
Summary of large-panel neutralization results (IC50)

| | | N(100f)Y^{1, 3} | DU025 | PG9RSH | DU303 | PGT145 | PGDM1400 |
|---|---|---|---|---|---|---|---|
| #VS Assayed | | 208 | 208 | 208 | 208 | 208 | 208 |
| # | IC₈₀ 50 μg/mL | 152 | 156 | 154 | 141 | 124 | 153 |
| VS | IC₈₀ < 10 μg/mL | 142 | 146 | 143 | 129 | 107 | 149 |
| Neut. | IC₈₀ < 1.0 μg/mL | 129 | 130 | 110 | 113 | 81 | 131 |
| | IC₈₀ < 0.1 μg/mL | 84 | 92 | 56 | 72 | 45 | 91 |
| | IC₈₀ < 0.01 μg/mL | 22 | 31 | 15 | 29 | 10 | 34 |
| % | IC₈₀ 50 μg/mL | 73 | 75 | 74 | 68 | 60 | 74 |
| VS | IC₈₀ < 10 μg/mL | 69 | 70 | 69 | 62 | 51 | 72 |
| Neut. | IC₈₀ < 1.0 μg/mL | 62 | 63 | 53 | 54 | 39 | 63 |
| | IC₈₀ < 0.1 μg/mL | 40 | 44 | 27 | 35 | 22 | 44 |
| | IC₈₀ < 0.01 μg/mL | 11 | 15 | 7 | 14 | 5 | 16 |
| | Median IC₈₀ ² | 0.086 | 0.058 | 0.227 | 0.09 | 0.276 | 0.047 |
| | Mean IC₈₀¹ | 0.106 | 0.078 | 0.236 | 0.116 | 0.343 | 0.069 |

¹Geometric mean calculated for samples with IC₈₀ < 50 μg/mL.
² Median calculated for samples with IC₈₀ < 50 μg/mL.
³Variant of PG9RSH.

Strains with Positively Charged Side Chains at Env Residue 169 are Neutralized More Potently by DU303

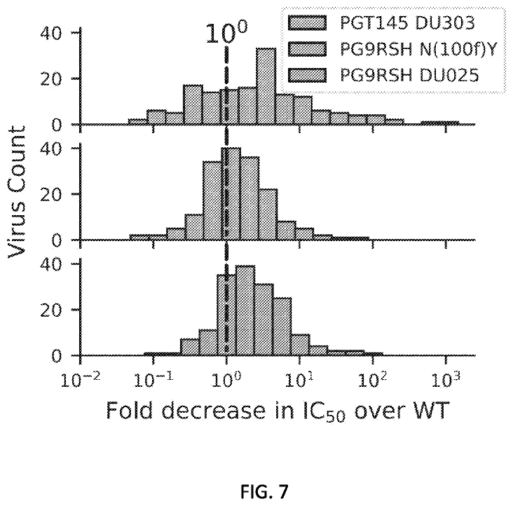

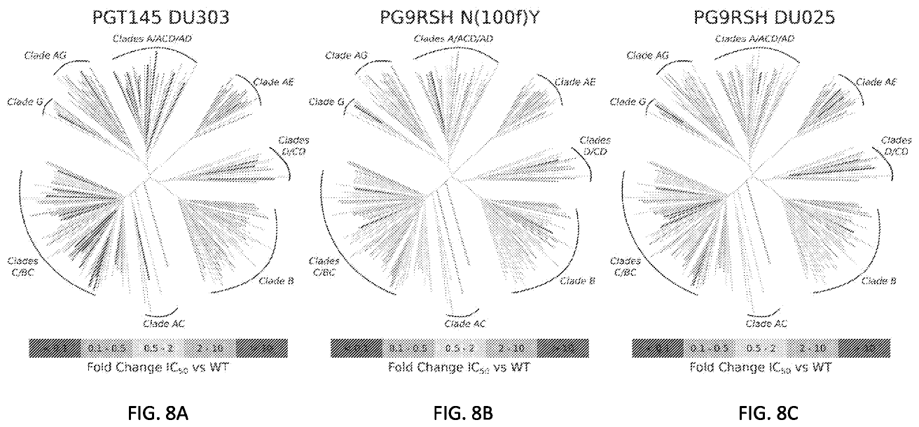

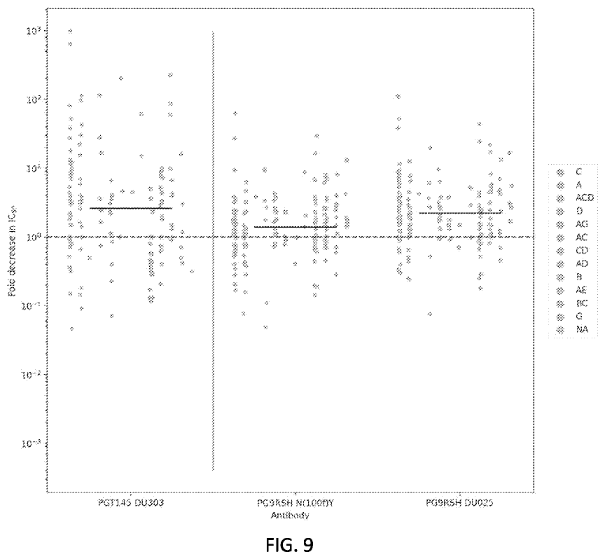

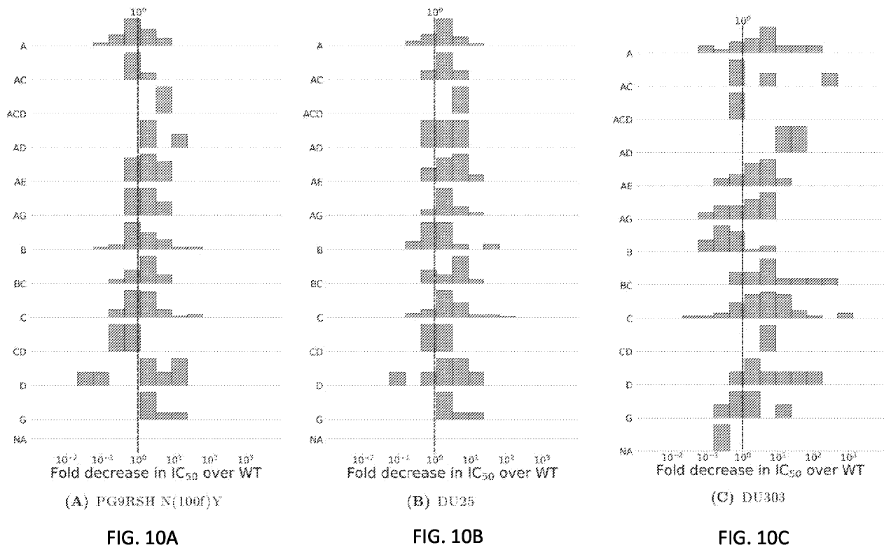

To further characterize the changes in neutralization activity for variant antibodies relative to PGT145 and PG9RSH, the fold decrease in IC₅₀ for each tested pseudovirus was computed (FIG. 7). Increases in potency of >100-fold were observed for seven virus-antibody pairs across five clades, including 988-, 643-, and 228-fold improvements in IC₅₀ for strains CAP256.206.C9, 16936-2.21, and CH038.12, respectively (Table 7). Analysis revealed a wide distribution of improvements in neutralization for DU303, with populations of strains with either slightly reduced or greatly improved neutralization. Conversely, both PG9RSH N(100f)Y and DU025 were characterized by a narrow distribution of fold decrease in IC₅₀ over tested pseudoviruses. The largest fold decrease in IC₅₀ for DU303 was 988-fold, occurring for the clade C pseudovirus CAP256.206.C9. The largest improvements in neutralization for PG9RSH N(100f)Y and DU025 were 63- and 110-fold, respectively, both occurring for the clade C pseudovirus ZM233.6. Examination of the fold change in neutralization by clade (FIGS. 8A, 9, and 10C) revealed the improvements in breadth and potency for DU303 to be non-uniform: marked improvements are evident for clades C/BC, A/AD, and D/CD, but decreases in neutralization occurred for clade B. On the other hand, the same analysis for both PG9RSH N(100f)Y (FIGS. 8B, 9, and 10A) and DU025 (FIGS. 8C, 9, and 10B) revealed most clades to show slight increases in neutralization with no clear pattern of decreased neutralization.

TABLE 7
Viruses with the largest differences in neutralization
(IC50) between wild-type and variant antibodies

| | | | WT | Variant | Fold- |
|---|---|---|---|---|---|
| Virus | Clade | Ab variant | IC₅₀ ¹ | IC₅₀ ¹ | change |
| ZM233.6 | C | PG9RSH DU025 | 0.11 | 0.001 | 110 |
| Q259.17 | A | PGT145 DU303 | 45.0 | 0.392 | 115 |
| 246-F3.C10.2 | AC | PGT145 DU303 | 17.4 | 0.086 | 202 |
| CH038.12 | BC | PGT145 DU303 | 46.5 | 0.204 | 228 |
| 16936-2.21 | c | PGT145 DU303 | 5.14 | 0.008 | 643 |
| CAP256.206.C9 | C | PGT145 DU303 | 3.95 | 0.004 | 988 |
| 191821.E6.1 | D | PGT145 DU303 | 3.35 | 0.029 | 116 |

¹ IC₅₀ values in μg/mL.

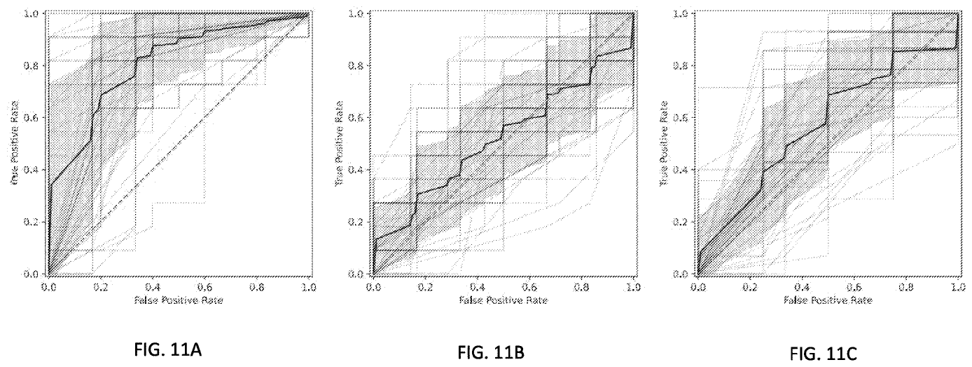

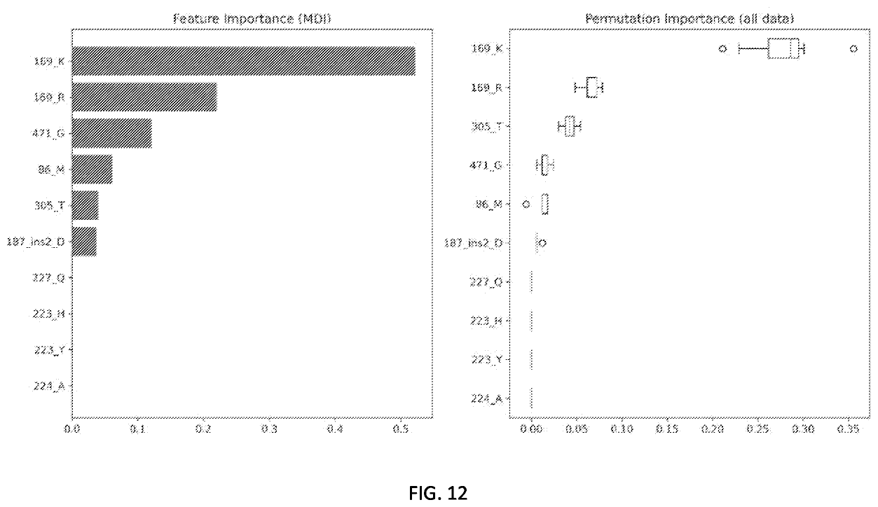

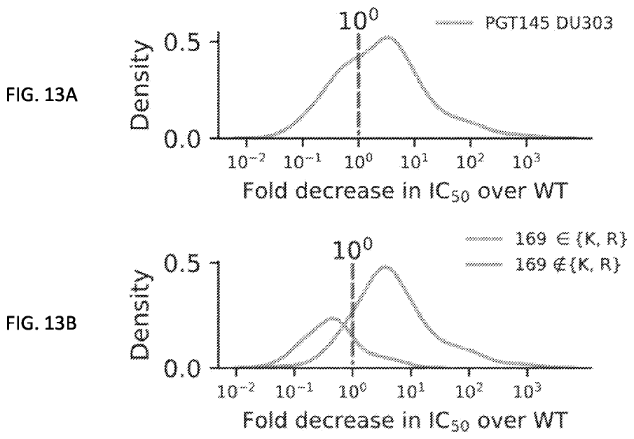

A gradient-boosted trees classifier predicted the changes in neutralization of pseudoviruses between wild-type PGT145 and DU303 and indicated these changes to be associated with the amino acid identity at Env residue 169. To investigate Env sequence features that may explain changes in neutralization for DU303, PG9RSH N(100f)Y, and DU025 relative to their ancestors, gradient-boosting tree models were trained to predict the sign of the change in neutralization for each variant based on pseudovirus Env sequences. Models were evaluated using repeated 10-fold nested cross-validation (Table 8; FIG. 11). The model for DU303 performed well, with a mean area under the curve (AUC) of 0.807±0.128, but models for PG9RSH N(100f)Y and DU025 performed poorly, with mean AUCs of 0.521±0.134 and 0.571±0.134, respectively. To identify residues on Env that were important for the improved neutralization of DU303, the permutation importance (PI) of Env residues was evaluated. K169 and R169 were significant (PI>0.05) features that were associated with an increase in neutralization for DU303 relative to PGT145 (FIG. 12). Separating the large-panel data by residue identity at Env residue 169 revealed two distinct populations of viruses (FIG. 13): Viruses with a positively charged residue at Env residue 169 were more potently neutralized by DU303 than by PGT145, while viruses without a positively charged residue at this position were less potently neutralized by DU303.

TABLE 8
Results of repeated, nested 10-fold CV.
Inner 10-fold cross-validation was performed to tune
hyperparameters, hyperparameter choices were evaluated by AUC.

| bNAb model | ROC AUC | Accuracy | F1 Score |
|---|---|---|---|
| DU303 | 0.807 ± 0.128 | 0.732 ± 0.086 | 0.823 ± 0.054 |
| PG9RSH N(100f)Y | 0.521 ± 0.134 | 0.596 ± 0.085 | 0.721 ± 0.076 |
| DU025 | 0.571 ± 0.134 | 0.767 ± 0.051 | 0.864 ± 0.038 |

### Cryo-EM Structures of BG505 DS-SOSIP.664 Bound by DU303, PG9RSH N(100f)Y, and DU025 Reveal Improved Side-Chain Interactions

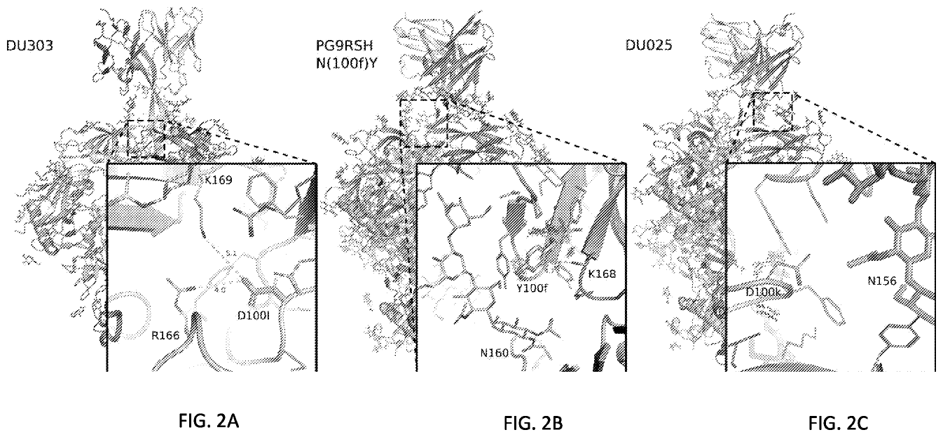

Cryo-EM structures of PGT145 variant DU303, PG9RSH N(100f)Y, and PG9RSH variant DU025 in complex with the BG505 DS-SOSIP.664 Env trimer were solved (FIG. 2). Three-dimensional reconstructions yielded resolutions of 3.58, 3.40, and 3.75 Å, respectively (Table 9), and local resolutions ranged between 3 and 9.3, 2.7 and 6.3, and 3.18 and 7.5 Å, respectively. The trimer apex and antibody CDRH3 loop were well resolved in all cases and indicated binding modes consistent with previous structures of PGT145 and PG9RSH (FIG. 2). These structures revealed details for key interactions between the HIV Env apex and the DU303, PG9RSH N(100f)Y, and DU025 variant antibodies.

TABLE 9
Cryo-EM data collection and refinement statistics

| | PG9RSH | PG9RSH | PGT145 |
|---|---|---|---|
| | N(100f)Y | DU025 BG505 | DU303 |
| | BG505 | IOS- | BG505 |
| | DS-SOSIP.664 | SOS1P.664 | DS-SOSIP.664 |
| EMDB ID | EMD-29248 | EMD-29264 | EMD-29288 |
| PDB ID | 8FK5 | 8FL1 | 8FLW |
| Microscope | FEI Titan | FEI Titan | FEI Titan |
| | Krios | Krios | Kilos |
| Voltage (kV) | 300 | 300 | 300 |
| Electron dose (e⁻ /Å²) | 63.75 | 63.75 | 63.75 |
| Detector | Gatan K3 | Gatan K3 | Gatan K3 |
| Pixel size (Å) | 1.083 | 1.083 | 1.083 |
| Defocus range (μm) | −0.8 to −2.5 | −0.8 to −2.5 | −0.8 to −2.5 |
| Magnification | 81,000 | 81,000 | 81,000 |
| Software | cryoSparc V3.1 | cryoSparc V3.1 | cryoSparc V3.1 |
| Particles | 230,180 | 104,665 | 107,753 |
| Symmetry | C1 | C1 | C1 |
| Box size (pix) | 340 | 340 | 340 |
| Resolution (Å) | 3.40 | 3.75 | 3.58 |
| (FSC_{0.143}) | | | |
| Software | Phenix 1.19 | Phenix 1.19 | Phenix 1.19 |
| Protein residues | 1,975 | 1,972 | 1,971 |
| Chimera CC | 78 | 79 | 76 |
| EMRinger score | 2.67 | 2.27 | 3.01 |
| R.M.S. deviations | | | |
| Bond lengths (Å) | 0.003 | 0.002 | 0.002 |
| Bond angles (°) | 0.469 | 0.460 | 0.538 |
| Molprobity score | 1.43 | 1.28 | 1.45 |
| Clash score | 3.76 | 3.90 | 3.37 |
| Favored rotamers (%) | 99.77 | 100 | 99.94 |
| Ramachandran | | | |
| Favored regions (%) | 96.12 | 97.47 | 95.33 |
| Disallowed | 0.05 | 0.00 | 0.00 |
| regions (%) | | | |

DU303 improves side-chain interactions with HIV Env residues 166 and 169 by introducing the N(100l)D mutation. Cryo-EM maps show well-resolved electron density for gp120 residues R166 and K169 but reveal ambiguity in the precise side-chain placements of residues D(100l) and F(100d). The atomic model of DU303 indicates that D(100l) could form electrostatic interactions with gp120 residues R166 and K169: the side-chain nitrogen of K169 lies 5.1 Å from a side-chain carboxyl oxygen of D(100l) (FIG. 3D). Similarly, one of the side-chain nitrogens of R166 lies 4 Å from a side-chain carboxyl oxygen of D(100l). The position of these side chains suggests that the negatively charged D(100l) forms favorable interactions with positively charged residues on gp120 to improve breadth and potency of neutralization.

PG9RSH N(100f)Y improves side-chain interactions with Env residue 168. Electron density maps show well-resolved density for gp120 residues D167, K168, and K169, along with the first two N-acetylglucosamine (GlcNAc) sugars of gp120 glycan N160. Density corresponding to bNAb residues is more ambiguous: peaks between the modeled side-chain locations of residues Y(100f) and Y(100a) suggest the presence of alternate rotamer configurations. Examination of low-density peaks (0.5° C.) reveals a small peak in density of the second GlcNAc of glycan N160 near the modeled location of Y(100f), suggesting interactions between Y(100f) and the glycan shield. The atomic model of PG9RSH N(100f)Y fit to the density map indicates that the primary interaction between Y(100f) and gp120 is a π-cation interaction with residue K168 (FIG. 3E). The ammonium nitrogen of K168 lies 4.7 Å from the center of the Y(100f) π system, and the angle between the distance vector and the ring normal vector is approximately 20°, which is representative of typical π-cation geometry. Y(100f) also forms van der Waals interactions with antibody residues P99, Y(100a), and TYS(100h). The side-chain geometry suggests that the aromatic Y(100f) side chain participates in a π-cation interaction with the positively charged K168 to improve potency of neutralization.

DU025 may improve long-range side-chain interactions or glycan interactions by introducing the Y(100k)D mutation. The electron density around the side chains of D(100k), Q170, K305, and Y173 are well resolved, along with the core of glycan N156. Interestingly, three unassigned density peaks arise in the groove between the V2 and V3 loops at both 1.2 and 3 σ, which could indicate the presence of solvent at this interface. Furthermore, a bridge of density at 1 σ arises between the modeled locations of Env residues Q170 and R308, hinting at long-range or solvent-mediated interactions. These data suggest that residue D(100k) may form long-range or solvent-mediated interactions with residues Q170 and K305, which lie at distances of 4.1 and 6.8 Å, respectively, in the atomic model.

### OSPREY Predictions are Validated by Cryo-EM Structures

OSPREY designs of antibody variants correctly predicted side-chain interactions. For DU303, predicted interaction distances between D(100l) and K169 and R169 in the OSPREY low-energy ensemble (LEE) differed by at most one angstrom from distances in the experimental model (FIGS. 3A and 3D). The side-chain orientations of this system were qualitatively similar between the LEE and the experimental model, indicating that OSPREY correctly predicted the structural consequences of the N(100l)D substitution. For PG9RSH N(100f)Y, side-chain locations in the LEE were qualitatively similar to those in the experimental model (FIGS. 3B and 3E). Interestingly, multiple rotamers of Y(100f) and Y(100a) appeared in the LEE, resulting in a conformation in which these side chains have rotated and stacked. The overall correspondence between the LEE and the experimental model indicated that OSPREY correctly predicted the structural consequences of the N(100f)Y substitution. Designs of DU025 predicted interactions with Q170 and R308, but differences in loop backbone conformation resulted in a change of environment near residue 100k. As a result, although the design ensemble correctly predicted that D(100k) creates long-range electrostatic interactions and correctly predicted a favorable interaction with Q170 (FIGS. 3C and 3F), the predicted interaction between D(100k) and R308 is not supported by the cryo-EM structure. Instead, shifts in the backbone create interactions between D(100k) and K305. However, the overall quality and type of interactions formed by D(100k) in the design ensemble were in fact consistent with the experimental structure.

### DISCUSSION

In this work, apex-directed anti-HIV bNAbs were designed for improved neutralization breadth using the OSPREY protein design software. The predicted affinity for the BG505 DS-SOSIP.664 trimer was used as a proxy for neutralization breadth during the design process. Assessment on a panel of 208 Env pseudoviruses indicated that three designed variants exhibited improved neutralization breadth and potency. Structures of these three variants bound to the BG505 DS-SOSIP.664 trimer were solved to investigate the mechanisms of improved neutralization potency. Relationships between Env epitope residue characteristics and neutralization potency were investigated to draw conclusions about mechanisms of improved breadth. Surprisingly, mutations that optimized interactions with variable epitope residues resulted in the largest improvements in breadth.

### Single Mutations Improve Antibody Neutralization of BG505

Experimental characterization of bNAb variants showed that DU303, PG9RSH N(100f)Y, and DU025 improved or maintained neutralization potency for the BG505 pseudovirus, with the most notable improvements observed by IC₈₀ measurements. Cryo-EM structures of each variant bound to the BG505 trimer indicate that OSPREY designs improved side-chain interactions. The PGT145 N(100l)D mutation (DU303) improved electrostatic interactions with the Env apex residues R166 and K169, improving charge complementarity. The PG9RSH N(100f)Y substitution created a π-cation interaction with Env residue K168 and may also interact with glycan N160. Finally, the PG9RSH Y(100k)D mutation (DU025) improved side-chain interactions with the polar Env residue Q170 and glycan N156. The interface around residue D(100k) is difficult to resolve, which may be indicative of a mobile or solvent-accessible environment. Unassigned electron-density peaks suggest that D(100k) may also form solvent-mediated interactions with K305 and perhaps even R308. All three designs were successful in improving neutralization potency against BG505 by optimizing side-chain interactions. The general correspondence between OSPREY-generated design ensembles and cryo-EM structures also indicated that the algorithms accurately modeled both environment and side-chain interactions at the PG9RSH and PGT145 epitopes. This is interesting given the low resolution of the design input structures and may be due to the fact that the algorithms are more sensitive to the input backbone conformation than the side chains, which are more difficult to resolve experimentally.

bNAb Variants Show Different Patterns of Improvement in Neutralization Breadth

The three best antibody variants improved neutralization breadth across a panel of 208 HIV pseudoviruses but differed in the pattern and extent of change in breadth. DU303 improved neutralization for most clades but sacrificed some subtype potency for clade B. PG9RSH N(100f)Y and DU025, on the other hand, increased neutralization in a relatively uniform manner across all clades. Interestingly, DU303 and DU025 improved overall breadth to a greater extent despite relatively low conservation of their Env epitope residues. Conversely, PG9RSH N(100f)Y resulted in smaller improvements despite the high conservation of Env residues that interact with the mutated antibody residue 100f.

PGT145 DU303 improved breadth by improving potency against “sensitive” strains containing a lysine or arginine at residue 169 while slightly decreasing potency against “resistant” strains with different substitutions at this epitope residue. Sensitive strains were more potently neutralized by ≈5-fold (geometric mean), while resistant strains were less potently neutralized by ≈2-fold (FIG. 13). Because the effect of improving neutralization against sensitive strains was larger than the effect of decreasing neutralization against resistant strains, and because there were more sensitive strains than resistant strains, the aggregate effect of the N(100l)D substitution was a gain of breadth and potency. These observations explain the loss of subtype potency for clade B, in which Env residue 169 is predominantly hydrophobic (valine, methionine) and unlikely to interact favorably with D(100l).

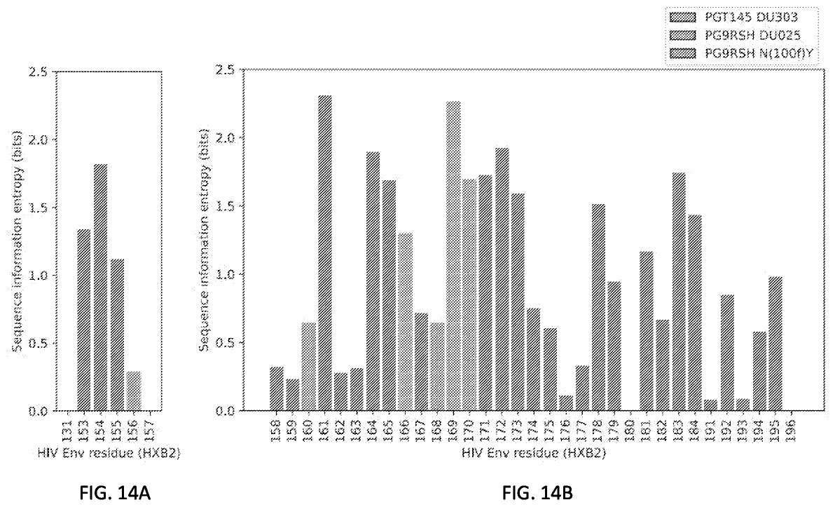

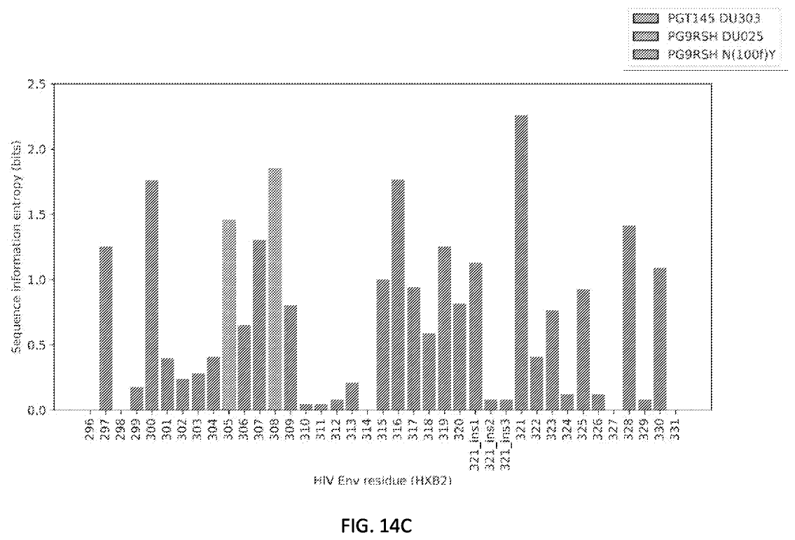

PG9RSH DU025 improved breadth of neutralization by improving interactions with variable residues on the Env V2 and V3 loops and with the conserved glycan N156. Analysis across a large panel of pseudoviruses revealed no major decreases in subtype potency, despite the relative variability of the epitope residues in proximity to residue D(100k) (FIG. 14). This could indicate that the Y(100k)D mutation improves breadth by improving interactions with the conserved glycan N156 or by improving interactions with the variable Env residues 170 and 305 in a manner that is tolerant of variation. Most panel strains have polar or charged residues at Env positions 170 (Q, K, or R) and 305 (K. R. or T), and it is possible that D(100k) interacts favorably with any of these amino acids, especially if interactions were to be solvent mediated.

PG9RSH N(100f)Y slightly improved breadth of neutralization by improving interactions with Env residue 168 and glycan N160. Overall, the slight improvement in breadth did not appear to sacrifice subtype potency, likely because the N(100f)Y substitution interacts with highly conserved Env features.

### Improvements in Breadth Did not Require Residue Conservation

These results presented herein demonstrate that antibody neutralization breadth can be increased by improving potency for a single “design antigen.” One intuitive explanation for this phenomenon is that the design antigen contains residues that are conserved across the entire antigen population. However, for the designs herein, epitope residue conservation did not appear to be critical for improving breadth. At least one variant (DU303) optimized interactions with epitope residues that were among the least conserved across the 208-strain test panel. Another (DU025) interacts with an epitope containing a conserved glycan, but structures suggest interactions with multiple non-conserved residues.

### Materials

TZM-bl cells are available through the NIH HIV Reagent Program, Division of AIDS, NIAID, NIH, contributed by Dr. John C. Kappes, Dr. Xiaoyun Wu and Tranzyme Inc.

### Experimental Model and Study Participant Details

These cells are a HeLa cell line generated from JC.53 cells that expresses CD4, CCR5, and CXCR4, with galactosidase and luciferase reporter genes under the HIV-1 promoter. For long-term storage store at or below −100° C. preferably in FBS supplemented with 40% DMEM and 10% DMSO. Propagate in DMEM supplemented with 10% FBS, 100 U per mL penicillin and 0.1 mg per mL streptomycin, incubate at 37° C.

### Redesign of PG9RSH and PGT145

Designs to obtain improved variants of PG9RSH and PGT145 were performed by defining sets of accessible conformations (conformation spaces) for unliganded antibody, unliganded Env trimer, and complexed antibody: Env states, followed by approximation of binding affinity using the K* algorithm (J. Comput. Biol., 12 (2005), pp. 740-761, 10.1089/cmb.2005.12.740; J. Comput. Chem., 29 (2008), pp. 1527-1542, 10.1002/jcc.20909; PLOS Comput. Biol., 8 (2012), p. e1002335) or an early version of the EWAK* algorithm (PLOS Comput. Biol., 16 (2020), Article e1007447) in OSPREY.

Conformation spaces were defined for PG9RSH or PGT145 based on an EM structure of the PG9 and 8ANC195 bNAbs in complex with the BG505 SOSIP.664 Env trimer (Protein Data Bank: 5VJ6) (Proc. Natl. Acad. Sci. USA, 110 (2013), pp. 4351-4356) or a Cryo-EM structure of PGT145 and sCD4 in complex with the DS-SOSIP.664 (Based on Protein Data Bank: 5U1F) (Nat. Struct. Mol. Biol., 24 (2017), pp. 370-378, respectively. The structure of PGT145 contained modeled locations of amino acid side chains and glycans, which were not present in the deposited structure due to resolution limitations.

Structures were inspected to determine whether they were suitable for design or needed refinement. MolProbity analysis (Protein Sci., 27 (2018), pp. 293-315) of the CDRH3 region of PG9 (5VJ6) revealed a few major clashes, one of which involved antibody backbone atoms, indicating that the atomic model may represent an inaccurate backbone conformation. As a result, the PG9 input structure was all-atom minimized using Yasara (Proteins: Struct., Funct., Bioinf., 77 (2009), pp. 114-122) to relax steric clashes, and the 8ANC195 antibody was removed along with some distal regions of gp160. This step was performed due to the very low resolution of the structure, coupled with the evident backbone clashes. Similar analysis of the PGT145 structure based on 5U1F (with added side chains) showed only a few clashes, each of which involved only side chain conformations. All-atom minimization of this structure was not performed, because we did not consider the relaxation of side chain conformations to be worth changing the backbone conformation without experimental evidence. The PGT145 input structure was modified by removing sCD4 along with parts of the PGT145 antibody and gp160 distal from peptide contacts of the PGT145: Env interface.

Hydrogens were added to both input structures using Reduce (J. Mol. Biol., 285 (1999), pp. 1735-1747). Backbone coordinates for the complex were defined by the resulting modified PG9: Env and PGT145: Env structures, and coordinates for the unliganded antibody and unliganded Env states were obtained by removing atoms corresponding to the Env and antibody, respectively. Design residues (See Table 1) were modeled as continuously flexible in OSPREY, for which rotamers from the Penultimate Rotamer Library (Proteins, 40 (2000), pp. 389-408) were allowed to adopt any side-chain conformation such that all χ-angles are within ±9° of their modal χ-angles. All other side-chain coordinates were obtained from the input structures. Rotamers and energetic interactions for sulfated tyrosines were modeled. A rotamer library was constructed, partial charges and force-field parameters were computed with Antechamber in AMBER and solvation parameters were computed using an extended version of the EEF1 solvation model. For each model ε-approximate bounds on the K* score were computed to a guaranteed accuracy of ε<0.683 using the K* or EWAK* algorithms.

### Antibody Variant Expression and Purification

DNA sequences of heavy and light chain variable regions of antibodies PG9RSH and PGT145 and variants were synthesized and subcloned into the pVRC8400 vector. For antibody expression, equal amounts of antibody heavy and light chain plasmid DNA were transfected into Expi293 cells using Turbo293 transfection reagent (Speed BioSystems). The transfected cells were incubated in shaker incubator at 120 rpm, 37° C., 9% CO2. The culture supernatants were harvested, filtered, and loaded on a protein A (GE Healthcare) column at 5 days post transfection. After washing the column with PBS, each antibody was eluted with an IgG elution buffer (Pierce) and immediately neutralized with one-tenth volume of 1M Tris-HCl pH 8.0. Eluted antibodies were dialyzed against PBS overnight and were confirmed by SDS-PAGE before use.

### Pseudovirus Neutralization Assays

Antibody neutralization was evaluated with the single-round infection assay of TZM-bl cells (J. Immunol. Methods, 409 (2014), pp. 131-146). Antibodies were serially diluted into wells of a 384-well plate, a constant amount of pseudovirus was added, plates were incubated for 60 min, and TZM-bl cells, which cells express luciferase upon viral infection, were added. Plates were incubated for 48 h, lysed, and measured for luciferase activity. The antibody concentration required to achieve 50% neutralization of infection (IC₅₀) was calculated using a dose-response curve fit with a 5-parameter nonlinear function. For small-panel neutralization assays a panel of 10 HIV-1 Env pseudoviruses from clades A, B, and C was used. For large-panel neutralization assays a panel of 208 geographically and genetically diverse HIV-1 Env pseudoviruses representing the major subtypes and circulating recombinant forms was used (Science, 340 (2013), pp. 751-756). All IC₅₀ values reported here are from small (10 viruses) or large (complete set of 208 viruses) neutralization panels run at the VRC. In some cases, multiple runs were averaged. Both the potency (measured as the median or geometric mean IC₅₀ or IC₈₀ for strains with measurable neutralization) and the breadth of neutralization (the number or percentage of strains with measurable neutralization) was evaluated. These summary statistics were computed in this way to conform to the literature standard and to enable straightforward comparison. Sources of error include the fact that neutralization IC₅₀ values can vary up to 3-fold between repeat assays.

### Cryo-EM Data Collection, Structure Determination, and Refinement

The BG505 DS-SOSIP.664 Env trimer was incubated with molar excess of antigen-binding fragment (Fab) for each of the improved V2-apex directed antibodies. Grids were prepared by depositing 2 μL of each complex at 2 mg/mL final concentration on C-flat 1.2/1.3 grids and vitrified with an FEI Vitrobot Mark IV with a wait time of 30 s, blot time of 3 s, and blot force of 1. Data collections were performed on a Titan Krios electron microscope with Leginon using a Gatan K3 direct detection device. Exposures were collected in movie mode for 2 s with the total dose of 63.75 e°Å/-Å2 fractionated over 40 raw frames. cryoSPARC v3.1 was used for frame alignment, CTF estimation, 2D classifications, ab initio 3D reconstruction, homogeneous refinement, and nonuniform 3D refinement. 3D reconstruction and final refinements were performed using C1 symmetry.

Coordinates from Protein Data Bank 5V8L and Protein Data Bank 3U4E were used for initial fits to the reconstructed maps. This was followed by simulated annealing and real space refinement in Phenix v1.19 with the sharpened map from cryoSPARC v3.1 and with a density modified map from Phenix Resolve and manually fit with Coot v0.9.8 and then improved through iterative rounds. Geometry and map fitting parameters were evaluated using Molprobity v4.5.1 and EMRinger. Maps and structures were deposited to the Electron Microscopy Data Bank (EMDB) (EMDB: EMD-29248, EMD-29264, EMD-29288) and PDB (PDB: 8FK5, 8FL1, 8FLW).

### Quantification and Statistical Analysis

Predicting Change in Neutralization from Env Sequence

Models were constructed using gradient-boosted decision trees in scikit-learn (J. Mach. Learn. Res., 12 (2011), pp. 2825-2830) which uses a boosting approach to construct ensemble models of CART decision trees.

To define labels corresponding to the change in neutralization relative to wild-type for each antibody the log-ratio of neutralization for each antibody (DU303, PG9RSH N(100f)Y, DU025) and its corresponding ancestor (PGT145, PG9RSH) was computed:

$$z = {log}_{10} \frac{{IC}_{50}^{WT}}{{IC}_{50}^{mut}}$$

Labels y were defined for binary classification where y=1 if z>0, y=0 otherwise. We processed Env protein alignments using BioPython (Bioinformatics, 25 (2009), pp. 1422-1423). To generate Env sequence features X the Env protein sequences were first augmented by identifying potential N-glycosylation sites, defined as sites containing the amino acid motif N-X-S/T, where X represents any amino acid. This resulted in 957 categorical features with an alphabet size of 21. Final features were obtained by one-hot encoding, resulting in a total of 4939 binary features.

For training three hyperparameters were optimized, leaving the rest at default values. An early stopping criterion implemented in sci-kit learn was used for training: 10% of the training data was held out as an additional validation set, and training was halted if the score on the validation set did not improve for a user-specified number of iterations. The maximum depth of the CART decision trees in the ensemble, the “learning rate”—a scaling of the contribution of each decision tree to the overall decision function, and the number of iterations of no improvement required for the early stopping criterion were optimized. Hyperparameters were optimized by 10-fold cross-validation (repeated 5 times) and parameters were selected by computing the average accuracy, AUC, or F1 score on the validation set.

Variable importance, measured by mean decrease in impurity (MDI) and permutation importance (PI), was evaluated for DU303 on a model trained using the entire available dataset. The MDI variable importance measure is analogous to the Gini importance—for each feature its MDI is defined as the average decrease in impurity over all nodes that correspond to the feature. In this case our splitting criterion is the Friedman Mean-Squared Error, Equation 35 in Friedman (Ann. Statist., 29 (2001), pp. 1189-1232). The MDI importance was computed using the scikit-learn implementation. PI was computed by randomly permuting each feature and then computing the difference in loss between using scrambled and original features using the scikit-learn implementation (sklearn.inspection.permutation_importance).

### Visualization and Figure Generation

Structure and density was visualized using PyMOL, USCF Chimera (J. Comput. Chem., 25 (2004), pp. 1605-1612) and King (Protein Sci., 18 (2009), pp. 2403-2409) and images were generated with PyMOL. Analysis of neutralization data was performed using Python, and accompanying figures were generated using the Matplotlib (Comput. Sci. Eng., 9 (2007), pp. 90-95) and Seaborn (J. Open Source Softw., 6 (2021), p. 3021,) libraries.

Sequences:

For the sequences below, CDRs are shown in bold. CDRH3 is shown in bold and underlined.
PG9 RSH heavy chain
ERLVESGGGVVQPGSSLRLSCAASGFDFSRQGMHWVRQAPGQGLEWVAFIKYDGSEK
YHADSVWGRLSISRDNSKDTLYLQMNSLRVEDTATYFCVREAGGPDYRNGYNYYDF
YDGYYNYHYMDVWGKGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVD
KKVEPKSCDKGLEVLFQ (SEQ ID NO: 1)
PG9 DU025 Light
QSALTQPASVSGSPGQSITISCQGTSNDVGGYESVSWYQQHPGKAPKVVIYDVSKRPSG
VSNRFSGSKSGNTASLTISGLQAEDEGDYYCKSLTSRSHRVFGTGTKLTVLGQPKAAPS
VTLFPPSSEELQANKATLVCLISDFYPGAVTVAWKADSSPVKAGVETTTPSKQSNNKYA
ASSYLSLTPEQWKSHRSYSCQVTHEGSTVEKTVAPTECS (SEQ ID NO: 2)
DU025 CDRS:
CDRH1-FDFSRQGMH (SEQ ID NO: 4)
CDRH2-FIKYDGSEKYHADSVWG (SEQ ID NO: 5)
CDRH3-EAGGPDYRNGYNYYDFDDGYYNYHYMDV (SEQ ID NO: 3)
CDRL1-QGTSNDVGGYESVS (SEQ ID NO: 6)
CDRL2-DVSKRPS (SEQ ID NO: 7)
CDRL3-KSLTSRSHRV (SEQ ID NO: 8)
PG9 DU025 Heavy
ERLVESGGGVVQPGSSLRLSCAASGFDFSRQGMHWVRQAPGQGLEWVAFIKYDGSEK
YHADSVWGRLSISRDNSKDTLYLQMNSLRVEDTATYFCVREAGGPDYRNGYNYYDF
DDGYYNYHYMDVWGKGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVD
KKVEPKSCDKGLEVLFQ (SEQ ID NO: 9)
PG9 DU011 Light Chain
QSALTQPASVSGSPGQSITISCQGTSNDVGGYESVSWYQQHPGKAPKVVIYDVSKRPSG
VSNRFSGSKSGNTASLTISGLQAEDEGDYYCKSLTSRSHRVFGTGTKLTVLGQPKAAPS
VTLFPPSSEELQANKATLVCLISDFYPGAVTVAWKADSSPVKAGVETTTPSKQSNNKYA
ASSYLSLTPEQWKSHKSYSCQVTHEGSTVEKTVAPTECS (SEQ ID NO: 10)
PG9 DU011 CDRS
CDRH1-FDFSRQGMH (SEQ ID NO: 4)
CDRH2-FIKYDGSEKYHADSVWG (SEQ ID NO: 5)
CDRH3-EAGGPDYRNGYYYYDFYDGYYNYHYMDV (SEQ ID NO: 11)
CDRL1-QGTSNDVGGYESVS (SEQ ID NO: 6)
CDRL2-DVSKRPS (SEQ ID NO: 7)
CDRL3-KSLTSRSHR (SEQ ID NO: 12)
PG9 DU011 Heavy Chain
ERLVESGGGVVQPGSSLRLSCAASGFDFSRQGMHWVRQAPGQGLEWVAFIKYDGSEK
YHADSVWGRLSISRDNSKDTLYLQMNSLRVEDTATYFCVREAGGPDYRNGYYYYDF
YDGYYNYHYMDVWGKGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVD
KKVEPKSCDKGLEVLFQ (SEQ ID NO: 13)
PGT145 WT heavy
QVQLVQSGAEVKKPGSSVKVSCKASGNSFSNHDVHWVRQATGQGLEWMGWMSHEG
DKTGLAQKFQGRVTITRDSGASTVYMELRGLTADDTAIYYCLTGSKHRLRDYFLYNE
YGPNYEEWGDYLATLDVWGHGTAVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKP
SNTKVDKKVEPKSCDKGLEVLFQ (SEQ ID NO: 14)
PGT145 DU303 Light
EVVITQSPLFLPVTPGEAASLSCKCSHSLQHSTGANYLAWYLQRPGQTPRLLIHLATHR
ASGVPDRFSGSGSGTDFTLKISRVESDDVGTYYCMQGLHSPWTFGQGTKVEIKRTVAA
PSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC (SEQ ID NO: 15)
DU303 CDRs
CDRH1-NSFSNHDVH (SEQ ID NO: 17)
CDRH2 MSHEGDKTGLAQKFQG (SEQ ID NO: 18)
CDRH3 GSKHRLRDYFLYNEYGPDYEEWGDYLATLDV (SEQ ID NO: 16)
CDRL1 SHSLQHSTGANYLA (SEQ ID NO: 19)
CDRL2 LATHRAS (SEQ ID NO: 20)
CDRL3 MQGLHSPWT (SEQ ID NO: 21)
DU303 HCDR3-SEQ ID NO: 11
PGT145 DU303 Heavy
QVQLVQSGAEVKKPGSSVKVSCKASGNSFSNHDVHWVRQATGQGLEWMGWMSHEG
DKTGLAQKFQGRVTITRDSGASTVYMELRGLTADDTAIYYCLTGSKHRLRDYFLYNE
YGPDYEEWGDYLATLDVWGHGTAVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKP
SNTKVDKKVEPKSCDKGLEVLFQ (SEQ ID NO: 22)

1. An anti-HIV-1 broadly neutralizing antibody (bNAb), the antibody comprising:
a heavy chain comprising a sequence having at least 80% sequence identity to SEQ ID NO: 1, wherein the heavy chain comprises a Y to D substitution mutation at position 114 relative to SEQ ID NO: 1, and
a light chain having at least 80% sequence identity to SEQ ID NO: 2.

2. The antibody of claim 1, wherein the heavy chain comprises a sequence having at least 90% sequence identity to SEQ ID NO: 1 and the light chain comprises a sequence having at least 90% sequence identity to SEQ ID NO: 2, or wherein the heavy chain comprises a sequence having at least 95% sequence identity to SEQ ID NO: 1 and the light chain comprises a sequence having at least 95% sequence identity to SEQ ID NO: 2.

3. (canceled)

4. The antibody of claim 1, wherein the heavy chain comprises a complementary determining region 3 (CDRH3) having a sequence of SEQ ID NO: 3.

5. The antibody of claim 4, wherein the heavy chain comprises complementary determining regions (CDRs) CDRH1, CDRH2, and CDRH3 comprising SEQ ID NO: 4, SEQ ID NO: 5, and SEQ ID NO: 3, respectively, and wherein the light chain comprises CDRS CDRL1, CDRL2, and CDRL3 comprising SEQ ID NO: 6, SEQ ID NO: 7, and SEQ ID NO: 8, respectively.

6. The antibody of claim 1, wherein the heavy chain comprises a sequence of SEQ ID NO: 9 and the light chain comprises a sequence of SEQ ID NO: 2.

7. An anti-HIV-1 broadly neutralizing antibody (bNAb), the antibody comprising:
a heavy chain comprising a sequence having at least 80% sequence identity to SEQ ID NO: 1, wherein the heavy chain comprises a N to Y substitution mutation at position 109 relative to SEQ ID NO: 1, and
a light chain having at least 80% sequence identity to SEQ ID NO: 10.

8. The antibody of claim 7, wherein the heavy chain comprises a sequence having at least 90% sequence identity to SEQ ID NO: 1 and the light chain comprises a sequence having at least 90% sequence identity to SEQ ID NO: 10, or wherein the heavy chain comprises a sequence having at least 95% sequence identity to SEQ ID NO: 1 and the light chain comprises a sequence having at least 95% sequence identity to SEQ ID NO: 10.

9. (canceled)

10. The antibody of claim 7, wherein the heavy chain comprises a complementary determining region 3 (CDRH3) having a sequence of SEQ ID NO: 11.

11. The antibody of claim 10, wherein the heavy chain comprises complementary determining regions (CDRs) CDRH1, CDRH2, and CDRH3 comprising SEQ ID NO: 4, SEQ ID NO: 5, and SEQ ID NO: 11, respectively, and wherein the light chain comprises CDRS CDRL1, CDRL2, and CDRL3 comprising SEQ ID NO: 6, SEQ ID NO: 7, and SEQ ID NO: 12, respectively.

12. The antibody of claim 7, wherein the heavy chain comprises a sequence of SEQ ID NO: 13 and the light chain comprises a sequence of SEQ ID NO: 10.

13. An anti-HIV-1 broadly neutralizing antibody (bNAb), the antibody comprising:
a heavy chain comprising a sequence having at least 80% sequence identity to SEQ ID NO: 14, wherein the heavy chain comprises a N to D substitution mutation at position 116 relative to SEQ ID NO: 14, and
a light chain having at least 80% sequence identity to SEQ ID NO: 15.

14. The antibody of claim 13, wherein the heavy chain comprises a sequence having at least 90% sequence identity to SEQ ID NO: 14 and the light chain comprises a sequence having at least 90% sequence identity to SEQ ID NO: 15, or wherein the heavy chain comprises a sequence having at least 95% sequence identity to SEQ ID NO: 14 and the light chain comprises a sequence having at least 95% sequence identity to SEQ ID NO: 15.

15. (canceled)

16. The antibody of claim 13, wherein the heavy chain comprises a complementary determining region 3 having a sequence of SEQ ID NO: 16.

17. The antibody of claim 16, wherein the heavy chain comprises complementary determining regions (CDRs) CDRH1, CDRH2, and CDRH3 comprising SEQ ID NO: 17, SEQ ID NO: 18, and SEQ ID NO: 16, respectively, and wherein the light chain comprises CDRS CDRL1, CDRL2, and CDRL3 comprising SEQ ID NO: 19, SEQ ID NO: 20, and SEQ ID NO: 21, respectively.

18. The antibody of claim 13, wherein the heavy chain comprises a sequence of SEQ ID NO: 22 and the light chain comprises a sequence of SEQ ID NO: 15.

19. A method of treating a viral infection in a subject, comprising administering to the subject the antibody of claim 1.

20. The antibody of claim 19, wherein the viral infection is an HIV infection.

21. A method of treating a viral infection in a subject, comprising administering to the subject the antibody of claim 7.

22. A method of treating a viral infection in a subject, comprising administering to the subject the antibody of claim 13.
